(19) United States
(12) Patent Application Publication
(10) Pub. No.: US 2024/0191213 A1
(43) Pub. Date: Jun. 13, 2024

(54) GENE THERAPIES FOR 21-HYDROXYLASE DEFICIENCY

(71) Applicant: Adrenas Therapeutics, Inc., Palo Alto, CA (US)

(72) Inventors: Clayton BEARD, Durham, NC (US); Kamal BHARUCHA, Dublin, CA (US); Pierre BOUGNERES, Le Kremlin-Bicetre (FR); Eric DAVID, Sante Fe, CA (US); Rachel ECLOV, Cary, NC (US); Rafael ESCANDON, Bainbridge Island, WA (US); Genevieve LAFORET, Bolton, MA (US); Adam SHAYWITZ, Irvine, CA (US); Sophie LE FUR, Vincennes (FR)

(21) Appl. No.: 18/551,143

(22) PCT Filed: Mar. 18, 2022
(86) PCT No.: PCT/US2022/020933

§ 371 Date: Sep. 18, 2023

Related U.S. Application Data
(60) Provisional application No. 63/163,634, filed on Mar. 19, 2021.

(51) Int. Cl. C12N 9/02; A61K 31/436; A61K 31/573; A61K 38/44; A61K 48/00; A61P 5/40; A61P 5/44; C12N 15/86

(52) U.S. Cl. CPC C12N 9/0071; A61K 31/436; A61K 31/573; A61K 48/005; A61P 5/40; A61P 5/44; C12N 15/86; A61K 38/44; C12N 2750/14143; C12N 2800/22; C12Y 114/14

(57) ABSTRACT

Disclosed herein are recombinant adeno-associated viral vectors expressing 21-hydroxy lase (21OH) protein and related uses for treating 21OH deficiency.

### CROSS REFERENCE TO RELATED APPLICATIONS

The present Application claims the benefit of priority to U.S. Provisional Application No. 63/163,634, filed on Mar. 19, 2021, the contents of which are hereby incorporated by reference in their entirety.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: ADRE_002_01 WO_SL, date recorded: Mar. 17, 2022, file size: 28.5 kilobytes).

### TECHNICAL FIELD

The present disclosure relates generally to the field of gene therapy. In particular, the disclosure describes recombinant adeno-associated virus (rAAV) vectors and particles that express 21-hydroxylase (21OH) protein. The rAAV vectors and particles may be used to treat 21OH deficiency.

### BACKGROUND

21-hydroxy lase (21OH) is a cytochrome P450 enzyme, encoded by the CYP21A2 gene, that is involved with the biosynthesis of the steroid hormones aldosterone and cortisol. These syntheses take place in the adrenal cortex. The high rate of recombination between the functional CYP21A2 gene and the closely linked, non-functional CYP21A1P pseudogene results in the relatively high incidence of congenital adrenal hyperplasia (CAH) and its unusual genetics, driven by gene conversions rather than by point mutations. Defects in CYP21A2 cause 21-hydroxylase deficiency (21OHD), which leads either to i) CAH with fetal virilization of female external genitalia, low or absent glucocorticoid and mineralocorticoid production, and large excess of androgens (“classical” 21OHD) or ii) milder forms of the disease without masculinization, without clinically meaningful cortisol and aldosterone deficits, but with increased production of androgens (“non-classical” 21OHD).

After decades of therapeutic strategies, management of severe forms of 21OHD remains clinically challenging. While patients can be treated with exogenous steroids, infant and adult patients remain at risk for adrenal crisis—the inability of their adrenal glands to respond to bodily stress such as routine infection, trauma, or intense exertion. Adrenal crisis can lead rapidly to severe shock and death even in well-educated patients who are compliant with therapy. See, Hahner et al., J Clin Endocrinol Metab, Feb: 100(2): 407-416 (2015). Additionally, there are significant consequences related to growth, gender, and sexuality. In female patients, there is an inherent difficulty of suppressing adrenal androgen production using supra-physiological glucocorticoid doses. As a result, alternating cycles of androgen versus glucocorticoid excess may lead to short stature, obesity, repeated genital surgery during childhood, alterations in puberty and chronic virilization. Hyperandrogenism remains the main cosmetic burden for female patients affected with classical and non-classical forms of the disease through hirsutism, male muscular development, enlarged clitoris size and impaired sexuality. See, Gastaud et al., J Clin Endocrinol Metab, 92(4), 1391-1396 (2007). Male patients are at risk for short stature and premature virilization. Therapeutic failure may even lead to bilateral adrenalectomy in some patients (Gmyrek et al., Pediatrics, 109: E28 (2002); Bruining et al., J Clin Endocrinol Metab, 86: 482-484 (2001)).

There remains a need for therapies that allow for persistent correction of 21OHD.

### SUMMARY

The present disclosure provides methods and compositions for use in the treatment of hormone imbalance and, in particular, 21-hydroylase (21OH) deficiency (21OHD), such as congenital adrenal hyperplasia (CAH). The methods provided herein comprise administration of viral vectors including nucleic acid molecules encoding a 21OH protein.

In an aspect, the present disclosure provides a method comprising: administering to a subject a therapeutically effective amount of a recombinant adeno-associated virus (rAAV) vector, wherein the rAAV vector comprises: (i) a nucleic acid molecule comprising at least one AAV inverted terminal repeat (ITR) and (ii) a non-AAV nucleotide sequence encoding a 21-hydroxy lase (21OH) protein, wherein the non-AAV nucleotide sequence is operably linked to a promoter; and wherein the therapeutically effective amount is in the range of about 10¹² vector genomes per kilogram (vg/kg) to about 10¹⁴ vg/kg.

In some embodiments, the subject is in need of expression of 21OH. In some embodiments, the subject has a 21OH deficiency (21OHD). In some embodiments, the subject is female and has ambiguous genitalia by Prader staging. In some embodiments, the subject has congenital adrenal hyperplasia (CAH). In some embodiments, the CAH is classical CAH. In some embodiments, administration of the rAAV vector results in expression of 21OH in the subject. In some embodiments, the 21OH is expressed in the subject's adrenal cortex, adrenal medulla, adrenal stem cells, adrenal progenitor cells, liver, or ovary.

In some embodiments, the subject is in need of cortisol and/or aldosterone. In some embodiments, the subject has a cortisol deficiency and/or an aldosterone deficiency. In some embodiments, the subject has an excess of progesterone, 17-OHP, renin, androstenedione (A4) and/or 11-ketosteroids. In some embodiments, the subject has an excess of progesterone, 17-OHP, renin, androstenedione (A4) and/or 11-ketosteroids in the blood and/or urine.

In another aspect, the present disclosure provides methods of expressing 21-hydroxlase (21OH) in a subject in need thereof. In some embodiments, such a method comprises: administering to the subject a therapeutically effective amount of a recombinant adeno-associated virus (rAAV) vector, wherein the rAAV vector comprises: (i) a nucleic acid molecule comprising at least one AAV inverted terminal repeat (ITR) and (ii) a non-AAV nucleotide sequence encoding a 21-hydroxylase (21OH) protein, wherein the non-AAV nucleotide sequence is operably linked to a promoter; and wherein the therapeutically effective amount is in the range of about 10¹² vg/kg to about 10¹⁴ vg/kg, thereby expressing 21OH in the subject.

In some embodiments, the 21OH is expressed in the subject's adrenal cortex, adrenal medulla, adrenal stem cells, adrenal progenitor cells, liver, or ovary.

### BRIEF DESCRIPTION OF THE DRAWINGS

FIG. 1A depicts the increase in weight gain in mice treated with the indicated doses of AAV5 encoding a 21-hydroxylase (21OH) protein over time, compared to untreated control mice. FIG. 1B depicts the dose-dependent increase in the viral genome of AAV5 encoding a 21-hydroxylase (21OH) protein detected in the adrenal gland of model mice (triangles) or non-human primates (NHPs, squares). FIG. 1C shows amount of human 21-OH produced, expressed as percentage of endogenous 21-OH in the adrenal gland of model mice (triangles) or non-human primates (NHPs).

FIG. 2 shows the timeline for a Phase 1/2, first in-human, open-label, dose-escalation study in adults with classical CAH due to 21-OHD.

FIG. 3 shows timeline for potential dose escalation and expansion in the Phase 1/2 clinical study

FIG. 4 shows the design of the AAV-hCYP21-Opt vector, the active ingredient in BBP-631, which comprises the following elements from 5′ to 3″: AAV 5′ ITR, the AAV packaging signal (Ψ), cytomegalovirus enhancer, chicken β-actin promoter including the canonical Kozak sequence with splice donor and rabbit β-globin intron with splice acceptor, the codon optimized human 21-hydroxylase (21-OH) cDNA, rabbit β-globin poly A signal, and the AAV 3′ ITR.

### DETAILED DESCRIPTION

The present disclosure relates to recombinant adeno-associated virus (AAV) vectors that are engineered to express 21-hydroxylase (21OH) and can be used to treat 21-hydroxylase deficiency (21OHD) including congenital adrenal hyperplasia (CAH). In some aspects, the present disclosure provides methods of treatment, expression of 21OH, and/or hormonal adjustment, and compositions for use in the same, comprising administration of a recombinant AAV vector comprising a non-AAV nucleotide sequence encoding a 21-OH protein.

The current approach to the clinical management for CAH is to treat with exogenous glucocorticoids (and mineralocorticoids, if necessary) to address the primary adrenal insufficiency and to normalize adrenal androgens, which often requires supraphysiological doses of glucocorticoids. Because supraphysiologic doses of glucocorticoids are often required to adequately control the levels of adrenal androgens, lifelong glucocorticoid replacement in CAH is associated with significant morbidities including reduced overall lifespan, cardiovascular disease, metabolic disease, bone disease, and short stature. Additionally, in classical CAH patients with a salt-wasting phenotype, lifelong mineralocorticoid replacement is also required.

In addition to androstenedione (A4), other adrenal hormones, in particular 17-OHP, are often used to guide management as well. Although the clinical management paradigm may seem straightforward, clinicians face an exceedingly difficult balancing act in adjusting maintenance glucocorticoid dosing in both children and adults with CAH. For example, clinicians attempt to treat children with the lowest, safest possible maintenance dose of glucocorticoids, particularly to preserve growth potential and final adult height. Higher, supraphysiologic doses of glucocorticoid replacement therapy may help protect both children and adult patients from symptoms related to hyperandrogenism, but also carry the risk of a myriad of long-term metabolic, skeletal, and cardiovascular sequelae. As CAH patients are on exogenous glucocorticoid and/or mineralocorticoid since infancy, the current treatment approach places a high care burden on patients and caretakers, predisposing to non-compliance with current standard of care.

There are substantial morbidities associated with the disease and current standard of care, including reduced overall lifespan, cardiovascular disease, metabolic disease, bone disease, and short stature. Furthermore, adrenal crisis can lead rapidly to severe shock and death and requires significant patient and caregiver education regarding stress dosing of glucocorticoids. The lifelong burden of disease remains high among classical CAH patients on the current standard of care, including 5 fold higher all-cause mortality. Thus, alternative therapies that obviate the need for long-term supraphysiologic glucocorticoids and have the potential to restore endogenous cortisol production are highly desirable for all classical CAH patients.

The present disclosure provides recombinant adeno-associated virus (AAV) vectors that are engineered to express 21-hydroxylase (21OH) and can be used to treat 21-hydroxylase deficiency (21OHD). In some aspects, the present disclosure provides a recombinant adeno-associated virus (rAAV) vector comprising a non-AAV nucleotide sequence encoding a 21OH protein, an rAAV particle comprising such a vector and methods of using such vectors and particles to treat 21OHD in subjects in need thereof.

In some embodiments, the compositions and methods disclosed herein restore adrenocortical cell function by providing an endogenous and physiologic pathway for glucocorticoid and mineralocorticoid synthesis. Thus, in some embodiments, the compositions and methods disclosed herein reduce the reliance on exogenous glucocorticoids (such as, hydrocortisone) and/or mineralocorticoids for disease management and thereby significantly reduce adverse effects from either excessive glucocorticoid replacement or excessive levels of serum androgens. In some embodiments, the compositions and methods disclosed herein alleviate at least one symptom or clinical feature of CAH associated with high levels of precursors such as 17-OHP and androgens such androstenedione (A4) and 11-oxo or 11-keto sterols (e.g., 11-keto-testosterone). Thus, in some embodiments, the compositions and methods disclosed herein alleviate hyperandrogenism associated with 21-OHD. In some embodiments, the compositions and methods disclosed herein reduce the risk of life-threatening adrenal crises.

The section headings used herein are for organizational purposes only and are not to be construed as limiting the subject matter described. All documents, or portions of documents, cited herein, including but not limited to patents, patent applications, articles, books, and treatises, are hereby expressly incorporated by reference in their entirety for any purpose. In the event that one or more of the incorporated documents or portions of documents define a term that contradicts that term's definition in the application, the definition that appears in this application controls. However, mention of any reference, article, publication, patent, patent publication, and patent application cited herein is not, and should not be taken as an acknowledgment, or any form of suggestion, that they constitute valid prior art or form part of the common general knowledge in any country in the world.

In the present description, any concentration range, percentage range, ratio range, or integer range is to be understood to include the value of any integer within the recited range and, when appropriate, fractions thereof (such as one tenth and one hundredth of an integer), unless otherwise indicated. The term “about”, when immediately preceding a number or numeral, means that the number or numeral ranges plus or minus 10%. It should be understood that the terms “a” and “an” as used herein refer to “one or more” of the enumerated components unless otherwise indicated. The use of the alternative (e.g., “or”) should be understood to mean either one, both, or any combination thereof of the alternatives. The term “and/or” should be understood to mean either one, or both of the alternatives. As used herein, the terms “include” and “comprise” are used synonymously.

### Recombinant AAV Vectors and Particles

In one aspect, the present disclosure provides a viral vector for delivery of a 21-hydroxylase (21OH) nucleic acid sequence to cells in need of treatment. Thus, in one embodiment, the present disclosure relates to a recombinant adeno-associated virus (rAAV) vector comprising a nucleic acid molecule comprising at least one AAV inverted terminal repeat (ITR) and a non-AAV nucleotide sequence (also referred to as a heterologous polynucleotide) encoding a 21OH protein, the non-AAV nucleotide sequence operably linked to a promoter. As used herein, the term “operable linkage” or “operably linked” refers to a physical or functional juxtaposition of the components so described as to permit them to function in their intended manner. In the example of an expression control element (such as a promoter or enhancer) in operable linkage with a polynucleotide, the relationship is such that the control element modulates expression of the nucleic acid. More specifically, for example, two DNA sequences operably linked means that the two DNAs are arranged (cis or trans) in such a relationship that at least one of the DNA sequences is able to exert a physiological effect upon the other sequence. “Operably linked” may mean that the nucleic acid sequences being linked are contiguous, or substantially contiguous, and, where necessary to join two protein coding regions, contiguous and in reading frame.

In some embodiments, an rAAV vector expresses a 21OH protein that is a human 21OH protein. The CYP212 gene encodes 21OH protein. As used herein, 21OH may refer to the 21OH protein or nucleic acid sequence encoding said protein. In some cases, the 21OH protein expressed by an rAAV vector described herein is a native (e.g., wild-type) 21OH protein. The 21OH protein or polypeptide encoded by the nucleotide sequence includes full-length native sequences, as with a naturally occurring 21OH protein, as well as functional subsequences, modified forms or sequence variants so long as the subsequence, modified form or variant retains some degree of functionality of the native full-length 21OH protein. In methods and uses of the present disclosure, 21OH proteins and polypeptides encoded by the nucleotide sequences in an rAAV vector can be, but are not required to be, identical to the endogenous 21OH protein that is defective, or whose expression is insufficient, or deficient in the treated subject.

In some embodiments, the non-AAV nucleotide sequence (e.g., heterologous sequence) encoding a 21OH protein is the wild-type CYP21 gene sequence. In some embodiments, the non-AAV nucleotide sequence (e.g., heterologous sequence) encoding a 21OH protein has been codon-optimized with respect to the wild-type CYP21 gene sequence. In some embodiments, a 21OH-encoding nucleotide sequence of the present disclosure is a codon-optimized sequence and comprises or consists of SEQ ID NO: 50 (see Table 1). In some embodiments, a 21OH-encoding nucleotide sequence is at least 90%, at least 91%, at least 92%, at least 93%, at least 94%, at least 95%, at least 96%, at least 97%, at least 98%, or at least 99% identical to SEQ ID NO: 50.

Codon optimization takes advantage of redundancies in the genetic code to enable a nucleotide sequence to be altered while maintaining the same amino acid sequence of the encoded protein. In some embodiments, codon optimization is carried out to facilitate an increase or decrease in the expression of an encoded protein. This is effected by tailoring codon usage in a nucleotide sequence to that of a specific cell type, thus taking advantage of cellular codon bias corresponding to a bias in the relative abundance of particular tRNAs in the cell type. By altering the codons in the nucleotide sequence so that they are tailored to match the relative abundance of corresponding tRNAs, it is possible to increase expression. Conversely, it is possible to decrease expression by selecting codons for which the corresponding tRNAs are known to be rare in the particular cell type.

In some embodiments, a codon-optimized nucleotide sequence encoding a 21OH protein is more stable than the wild-type cDNA sequence, thereby avoiding generating alternatively spliced variants or truncated proteins if the non-AAV nucleotide sequence is introduced into the transcriptional machinery through gene therapy.

In some embodiments, the non-AAV nucleotide sequence (e.g., heterologous sequence) encoding a 21OH protein encodes the amino acid sequence of SEQ ID NO:1 (see Table 1). In other embodiments, the non-AAV nucleotide sequence encoding a 21OH protein encodes an amino acid sequence at least 90%, at least 91%, at least 92%, at least 93%, at least 94%, at least 95%, at least 96%, at least 97%, at least 98%, or at least 99% identical to SEQ ID NO:1.

TABLE 1
Non-limiting examples of 21OH (CYP21A2) and promoter sequences.

| Sequence | | SEQ |
|---|---|---|
| description | Sequence | ID NO |
| CYP21A2, | MLLLGLLLLLPLLAGARLLWNWWKLRSLHLPPLAPGFLHLLQPDLPIYLLGLTQK | 1 |
| Homo sapiens, | FGPIYRLHLGLQDVVVLNSKRTIEEAMVKKWADFAGRPEPLTYKLVSRNYPDLSL | |
| NCBI | GDYSLLWKAHKKLTRSALLLGIRDSMEPVVEQLTQEFCERMRAQPGTPVAIEEEF | |
| Reference | SLLTCSIICYLTFGDKIKDDNLMPAYYKCIQEVLKTWSHWSIQIVDVIPFLRFFP | |
| Sequence | NPGLRRLKQAIEKRDHIVEMQLRQHKESLVAGQWRDMMDYMLQGVAQPSMEEGSG | |
| NP_000491.4 | QLLEGHVHMAAVDLLIGGTETTANTLSWAVVFLLHHPEIQQRLQEELDHELGPGA | |
| | SSSRVPYKDRARLPLLNATIAEVLRLRPVVPLALPHRTTRPSSISGYDIPEGTVI | |
| | IPNLQGAHLDETVWERPHEFWPDRFLEPGKNSRALAFGCGARVCLGEPLARLELF | |
| | VVLTRLLQAFTLLPSGDALPSLQPLPHCSVILKMQPFQVRLQPRGMGAHSPGQSQ | |
| CAG | GCGTTACATAACTTACGGTAAATGGCCCGCCTGGCTGACCGCCCAACGACCCCCG | 2 |
| promoter | CCCATTGACGTCAATAATGACGTATGTTCCCATAGTAACGCCAATAGGGACTTTC | |
| | CATTGACGTCAATGGGTGGAGTATTTACGGTAAACTGCCCACTTGGCAGTACATC | |
| | AAGTGTATCATATGCCAAGTACGCCCCCTATTGACGTCAATGACGGTAAATGGCC | |
| | CGCCTGGCATTATGCCCAGTACATGACCTTATGGGACTTTCCTACTTGGCAGTAC | |
| | ATCTACGTATTAGTCATCGCTATTACCATGGTCGAGGTGAGCCCCACGTTCTGCT | |
| | TCACTCTCCCCATCTCCCCCCCCTCCCCACCCCCAATTTTGTATTTATTTATTTT | |
| | TTAATTATTTTGTGCAGCGATGGGGGCGGGGGGGGGGGGGGGGCGCGCGCCAGGC | |
| | GGGGCGGGGCGGGGCGAGGGGCGGGGCGGGGCGAGGCGGAGAGGTGCGGCGGCAG | |
| | CCAATCAGAGCGGCGCGCTCCGAAAGTTTCCTTTTATGGCGAGGCGGCGGCGGCG | |
| | GCGGCCCTATAAAAAGCGAAGCGCGCGGCGGGCG | |
| PGK promoter | CCGGTAGGCGCCAACCGGCTCCGTTCTTTGGTGGCCCCTTCGCGCCACCTTCTAC | 3 |
| | TCCTCCCCTAGTCAGGAAGTTCCCCCCCGCCCCGCAGCTCGCGTCGTGCAGGACG | |
| | TGACAAATGGAAGTAGCACGTCTCACTAGTCTCGTGCAGATGGACAGCACCGCTG | |
| | AGCAATGGAAGCGGGTAGGCCTTTGGGGCAGCGGCCAATAGCAGCTTTGCTCCTT | |
| | CGCTTTCTGGGCTCAGAGGCTGGGAAGGGGTGGGTCCGGGGGCGGGCTCAGGGGC | |
| | GGGCTCAGGGGCGGGGCGGGCGCCCGAAGGTCCTCCGGAGGCCCGGCATTCTGCA | |
| | CGCTTCAAAAGCGCACGTCTGCCGCGCTGTTCTCCTCTTCCTCATCTCCGGGCCT | |
| | TTCGACCTGCAGCC | |
| CB6 Promoter | CCACGTTCTGCTTCACTCTCCCCATCTCCCCCCCCTCCCCACCCCCAATTTTGTA | 48 |
| | TTTATTTATTTTTTAATTATTTTGTGCAGCGATGGGGGCGGGGGGGGGGGGCGCG | |
| | CGCCAGGCGGGGCGGGGCGGGGCGAGGGGGGGGCGGGGCGAGGCGGAGAGGTGC | |
| | GGCGGCAGCCAATCAGAGCGGCGCGCTCCGAAAGTTTCCTTTTATGGCGAGGCGG | |
| | CGGCGGCGGCGGCCCTATAAAAAGCGAAGCGCGCGGCGGG | |
| CBA | TGGTCGAGGTGAGCCCCACGTTCTGCTTCACTCTCCCCATCTCCCCCCCCTCCCC | 49 |
| Promoter | ACCCCCAATTTTGTATTTATTTATTTTTTAATTATTTTGTGCAGCGATGGGGGCG | |
| | GGGGGGGGGGGGGGGCGCGCGCCAGGCGGGGCGGGGCGGGGCGAGGGGCGGGGCG | |
| | GGGCGAGGCGGAGAGGTGCGGCGGCAGCCAATCAGAGCGGCGCGCTCCGAAAGTT | |
| | TCCTTTTATGGCGAGGCGGCGGCGGCGGCGGCCCTATAAAAAGCGAAGCGCGCGG | |
| | CGGGCGG | |
| Codon | ATGCTGCTGCTGGGGCTGCTGCTGCTGCTGCCTCTGCTGGCTGGGGCTCGACTGC | 50 |
| Optimized | TGTGGAACTGGTGGAAACTGCGGTCCCTGCACCTGCCACCTCTGGCACCAGGCTT | |
| CYP21A2 | CCTGCACCTGCTGCAGCCAGACCTGCCCATCTACCTGCTGGGCCTGACCCAGAAG | |
| gene sequence | TTTGGCCCTATCTATAGGCTGCACCTGGGCCTGCAGGACGTGGTGGTGCTGAACT | |
| | CTAAGCGCACCATCGAGGAGGCCATGGTGAAGAAGTGGGCAGATTTCGCAGGCCG | |
| | GCCAGAGCCACTGACATACAAGCTGGTGAGCAGAAATTATCCTGACCTGTCCCTG | |
| | GGCGATTACTCTCTGCTGTGGAAGGCCCACAAGAAGCTGACAAGGAGCGCCCTGC | |
| | TGCTGGGCATCCGCGACTCCATGGAGCCAGTGGTGGAGCAGCTGACCCAGGAGTT | |
| | TTGCGAGAGGATGAGGGCACAGCCTGGAACACCAGTGGCCATCGAGGAGGAGTTC | |
| | AGCCTGCTGACCTGCTCCATCATCTGTTATCTGACATTTGGCGATAAGATCAAGG | |
| | ACGATAACCTGATGCCAGCCTACTATAAGTGTATCCAGGAGGTGCTGAAGACCTG | |
| | GAGCCACTGGAGCATCCAGATCGTGGACGTGATCCCCTTCCTGAGGTTCTTTCCT | |
| | AATCCAGGCCTGCGGAGACTGAAGCAGGCCATCGAGAAGAGGGATCACATCGTGG | |
| | AGATGCAGCTGAGGCAGCACAAGGAGTCCCTGGTGGCAGGACAGTGGAGGGACAT | |
| | GATGGATTACATGCTGCAGGGAGTGGCACAGCCATCTATGGAGGAGGGAAGCGGA | |
| | CAGCTGCTGGAGGGACACGTGCACATGGCAGCAGTGGATCTGCTGATCGGAGGAA | |
| | CCGAGACAACAGCCAACACACTGAGCTGGGCCGTGGTGTTTCTGCTGCACCACCC | |
| | TGAGATCCAGCAGCGGCTGCAGGAGGAGCTGGACCACGAGCTGGGACCTGGAGCA | |
| | AGCTCCTCTAGAGTGCCATACAAGGATCGGGCCAGACTGCCCCTGCTGAATGCCA | |
| | CCATCGCCGAGGTGCTGAGGCTGCGCCCCGTGGTGCCTCTGGCCCTGCCTCACAG | |
| | GACCACAAGACCAAGCTCCATCTCCGGCTATGACATCCCAGAGGGCACCGTGATC | |
| | ATCCCAAACCTGCAGGGAGCACACCTGGACGAGACAGTGTGGGAGCGGCCACACG | |
| | AGTTCTGGCCCGATAGATTTCTGGAGCCTGGCAAGAACAGCCGGGCCCTGGCCTT | |
| | CGGCTGCGGAGCCCGGGTGTGCCTGGGCGAGCCACTGGCCAGGCTGGAGCTGTTC | |
| | GTGGTGCTGACCCGCCTGCTGCAGGCCTTTACACTGCTGCCCTCCGGCGATGCCC | |
| | TGCCTTCTCTGCAGCCACTGCCTCACTGCTCCGTGATCCTGAAGATGCAGCCCTT | |
| | TCAGGTCCGCCTGCAGCCAAGGGGGATGGGGGCACATAGTCCAGGGCAGTCTCAG | |
| | TAA | |

In some embodiments, the non-AAV nucleotide sequence (e.g., heterologous sequence) encoding a 21OH protein is the human 21OH cDNA, optionally linked to a nucleotide sequence encoding a hemagglutinin (HA) tag. In certain cases, the non-AAV nucleotide sequence (e.g., heterologous sequence) encoding a 21OH protein is linked to a nucleotide sequence encoding a tag, for example hemagglutinin (HA), UA, cMyc, or any suitable tag. “CYPHA” may refer to a 21OH transgene fused to a sequence encoding an HA tag.

The terms “identity,” “homology,” and grammatical variations thereof, mean that two or more referenced entities are the same, when they are “aligned” sequences. Thus, by way of example, when two polypeptide sequences are identical, they have the same amino acid sequence, at least within the referenced region or portion. Where two polynucleotide sequences are identical, they have the same polynucleotide sequence, at least within the referenced region or portion. The identity can be over a defined area (region or domain) of the sequence. An “area” or “region” of identity refers to a portion of two or more referenced entities that are the same. Thus, where two protein or nucleic acid sequences are identical over one or more sequence areas or regions they share identity within that region. An “aligned” sequence refers to multiple polynucleotide or protein (amino acid) sequences, often containing corrections for missing or additional bases or amino acids (gaps) as compared to a reference sequence.

The identity can extend over the entire sequence length or a portion of the sequence. In particular aspects, the length of the sequence sharing the percent identity is 2, 3, 4, 5 or more contiguous polynucleotide or amino acids, e.g., at least 6, 7, 8, 9, 10, 11, 12, 13, 14, 15, 16, 17, 18, 19, 20, or more contiguous amino acids. In additional particular aspects, the length of the sequence sharing identity is 20 or more contiguous polynucleotide or amino acids, e.g., at least 20, 21, 22, 23, 24, 25, 26, 27, 28, 29, 30, 31, 32, 33, 34, 35, or more contiguous amino acids. In further particular aspects, the length of the sequence sharing identity is 35 or more contiguous polynucleotide or amino acids, e.g., at least 35, 36, 37, 38, 39, 40, 41, 42, 43, 44, 45, 46, 47, 48, 49, 50, or more contiguous amino acids. In yet further particular aspects, the length of the sequence sharing identity is 50 or more contiguous polynucleotide or amino acids, e.g., at least 50-55, 55-60, 60-65, 65-70, 70-75, 75-80, 80-85, 85-90, 90-95, 95-100, 100-110, or more contiguous polynucleotide or amino acids.

The terms “homologous” or “homology” mean that two or more referenced entities share at least partial identity over a given region or portion. “Areas,” “regions,” or “domains” of homology or identity mean that a portion of two or more referenced entities share homology or are the same. Thus, where two sequences are identical over one or more sequence regions they share identity in these regions. “Substantial homology.” means that a molecule is structurally or functionally conserved such that it has or is predicted to have at least partial structure or function of one or more of the structures or functions (e.g., a biological function or activity) of the reference molecule, or relevant/corresponding region or portion of the reference molecule to which it shares homology.

The extent of identity (homology) between two sequences can be ascertained using a computer program and mathematical algorithm. Percentage identity can be calculated using the alignment program Clustal Omega, available at www.ebi.ac.uk/Tools/msa/clustalo using default parameters. See, Sievers et al., “Fast, scalable generation of high-quality protein multiple sequence alignments using Clustal Omega.” (2011 Oct. 11) Molecular systems biology 7:539. For the purposes of calculating identity to a sequence, extensions such as tags are not included.

Vector genome sequences, including rAAV vector genome sequences described herein, can include one or more “expression control elements,” Typically, expression control elements are nucleic acid sequences that influence expression of an operably linked polynucleotide. Control elements, including expression control elements as set forth herein, such as promoters and enhancers, present within a vector are included to facilitate proper heterologous polynucleotide (e.g., 21OH gene) transcription and/or translation (e.g., a promoter, enhancer, splicing signal for introns, maintenance of the correct reading frame of the gene to permit in-frame translation of mRNA, etc.). Expression control elements include appropriate transcription initiation, termination, promoter and enhancer sequences: efficient RNA processing signals such as splicing and polyadenylation (polyA) signals: sequences that stabilize cytoplasmic mRNA: sequences that enhance translation efficiency (i.e., Kozak consensus sequence): sequences that enhance protein stability; and, in some cases, sequences that enhance secretion of the encoded product (e.g., 21OH). In some embodiments, an rAAV vector genome sequence of the present disclosure comprises a consensus sequence such as a Kozak sequence (for example, a DNA sequence transcribed to an RNA Kozak sequence). In some embodiments, an rAAV vector genome sequence of the present disclosure comprises a Kozak sequence upstream of the nucleotide sequence encoding a 21OH protein. In some embodiments, an RNA Kozak sequence comprises or consists of ACCAUGG (SEQ ID NO:44), GCCGCCACCAUGG (SEQ ID NO:45), CCACCAUG (SEQ ID NO:46), or CCACCAUGG (SEQ ID NO:47).

Expression control can be carried out at the level of transcription, translation, splicing, message stability, etc. Typically, an expression control element that modulates transcription is juxtaposed near the 5′ end of the transcribed polynucleotide (i.e., “upstream”). Expression control elements can also be located at the 3′ end of the transcribed sequence (i.e., “downstream”) or within the transcript (e.g., in an intron). Expression control elements can be located at a distance away from the transcribed sequence (e.g., 100 to 500, 500 to 1000, 2000 to 5000, 5000 to 10,000, or more nucleotides from the nucleotide sequence expressing 21OH), even at considerable distances. Nevertheless, owing to the polynucleotide length limitations, for AAV vectors, such expression control elements will typically be within 1 to 1000 nucleotides from the nucleotide sequence encoding 21OH.

Functionally, expression of an operably linked nucleotide sequence encoding 21OH is at least in part controllable by the element (e.g., promoter) such that the element modulates transcription of the nucleotide sequence and, as appropriate, translation of the transcript. A specific example of an expression control element is a promoter, which is usually located 5′ of the transcribed sequence. Another example of an expression control element is an enhancer, which can be located 5′ of the transcribed sequence, 3′ of the transcribed sequence, or within the transcribed sequence.

A “promoter” as used herein can refer to a nucleic acid sequence that is located adjacent to a nucleic acid sequence (e.g., heterologous polynucleotide) that encodes a recombinant product (e.g., 21OH). A promoter is typically operably linked to an adjacent sequence, e.g., heterologous polynucleotide. A promoter typically increases an amount expressed from a heterologous polynucleotide as compared to an amount expressed when no promoter exists.

An “enhancer” as used herein can refer to a sequence that is located adjacent to a nucleotide sequence encoding 21OH. Enhancer elements are typically located upstream of a promoter element but also function and can be located downstream of or within a DNA sequence (e.g., a nucleotide sequence encoding 21OH). Hence, an enhancer element can be located, e.g., 100 base pairs, 200 base pairs, or 300 or more base pairs upstream or downstream of a heterologous polynucleotide. Enhancer elements typically increase expression of a heterologous polynucleotide above the level of increased expression afforded by a promoter element.

In some embodiments, expression control elements include ubiquitous, constitutive, or promiscuous promoters and/or enhancers which are capable of driving expression of a polynucleotide in many different cell types. Such elements include, but are not limited to, a cytomegalovirus/β-actin hybrid (e.g., CAG, CB6 or CBA) promoter, a phosphoglycerol kinase (PGK) promoter, cytomegalovirus (CMV) immediate early promoter and/or enhancer sequences, the Rous sarcoma virus (RSV) promoter and/or enhancer sequences and other viral promoters and/or enhancers active in a variety of mammalian cell types, or synthetic elements that are not present in nature (see, e.g., Boshart et al, Cell, 41:521-530 (1985)), the SV40 promoter, the dihydrofolate reductase promoter, the chicken β-actin (CBA) promoter, the EF 1 promoter (Invitrogen), the immediate early CMV enhancer coupled with the CBA promoter (Beltran et al., Gene Therapy, 17(9): 1162-1174 (2010)), and the CBh promoter (Gray et al., Hum Gene Ther, 22(9): 1143-1153 (2011)). In certain aspects, an rAAV of the present disclosure comprises a synthetic CASI promoter which contains a portion of the CMV enhancer, a portion of the chicken beta-actin promoter, and a portion of the UBC enhancer. See, e.g., WO 2012/115980. In some embodiments, an rAAV vector comprises a CAG promoter sequence comprising SEQ ID NO:2 or a nucleotide sequence at least 90%, at least 91%, at least 92%, at least 93%, at least 94%, at least 95%, at least 96%, at least 97%, at least 98% or at least 99% identical to SEQ ID NO:2. In some embodiments, an rAAV vector comprises a PGK promoter sequence comprising SEQ ID NO:3 or a nucleotide sequence at least 90%, at least 91%, at least 92%, at least 93%, at least 94%, at least 95%, at least 96%, at least 97%, at least 98% or at least 99% identical to SEQ ID NO:3. In some embodiments, an rAAV vector comprises a CB6 promoter sequence comprising SEQ ID NO:48 or a nucleotide sequence at least 90%, at least 91%, at least 92%, at least 93%, at least 94%, at least 95%, at least 96%, at least 97%, at least 98% or at least 99% identical to SEQ ID NO:48. In some embodiments, an rAAV vector comprises a CBA promoter sequence comprising SEQ ID NO:49 or a nucleotide sequence at least 90%, at least 91%, at least 92%, at least 93%, at least 94%, at least 95%, at least 96%, at least 97%, at least 98% or at least 99% identical to SEQ ID NO:49. See Table 1 for non-limiting examples of promoter sequences.

Inducible promoters allow regulation of gene expression and can be regulated by exogenously supplied compounds, environmental factors such as temperature, or the presence of a specific physiological state, acute phase, a particular differentiation state of the cell, or in replicating cells only. Inducible promoters and inducible systems are available from a variety of commercial sources, including, without limitation, Invitrogen and Clontech. Many other systems have been described and are available for use. Examples of inducible promoters regulated by exogenously supplied compounds, include, the zinc-inducible sheep metallothionine (MT) promoter, the dexamethasone (Dex)-inducible mouse mammary tumor virus (MMTV) promoter, the T7 polymerase promoter system: the ecdysone insect promoter, the tetracycline-repressible system, the tetracycline-inducible system, the RU486-inducible system and the rapamycin-inducible system. Any type of inducible promoter which is tightly regulated and is specific for the particular target cell type in which 21OH expression is intended may be used.

Expression control elements (e.g., promoters) include those active in a particular tissue or cell type, referred to herein as “tissue-specific expression control elements/promoters.” Tissue-specific expression control elements are typically active in specific cell or tissue (e.g., adrenal gland, adrenal cortex, liver, brain, central nervous system, spinal cord, eye, retina, bone, muscle, lung, pancreas, heart, kidney cell, etc.). Expression control elements are typically active in these cells, tissues or organs because they are recognized by transcriptional activator proteins, or other regulators of transcription, that are unique to a specific cell, tissue or organ type. Thus, in some cases, an rAAV vector of the present disclosure comprises a promoter that directs expression of the nucleotide sequence encoding 21OH protein in a host cell (e.g., an adrenal gland cell). In certain embodiments, an adrenal gland cell is an adrenal cortex cell. In some embodiments, an rAAV vector of the present disclosure comprises a non-AAV nucleotide sequence encoding a 21OH protein, the non-AAV nucleotide sequence operably linked to a promoter specific for expression in an adrenal cortex cell or an adrenal medulla cell. In some embodiments, an rAAV vector of the present disclosure comprises a non-AAV nucleotide sequence encoding a 21OH protein, the non-AAV nucleotide sequence operably linked to a promoter specific for expression in a subject's adrenal gland (e.g., adrenal cortex or adrenal medulla), liver or ovary. In certain embodiments, an rAAV vector of the present disclosure comprises a non-AAV nucleotide sequence encoding a 21OH protein, the non-AAV nucleotide sequence operably linked to a promoter specific for expression in an adrenal stem cell (e.g., an adrenocortical stem cell) or an adrenal progenitor cell.

In some embodiments, the regulatory sequences useful in the rAAV vectors of the present disclosure also contain an intron, which intron is optionally located between the promoter/enhancer sequence and the 21OH gene. In some embodiments, the intron sequence is derived from SV-40, and is a 100 bp mini-intron splice donor/splice acceptor referred to as SD-SA. In some embodiments, an rAAV vector comprises a posttranscriptional regulatory element. One example of a posttranscriptional regulatory element is the woodchuck hepatitis virus post-transcriptional element (WPRE). (See, e.g., Wang and Verma, Proc. Natl. Acad. Sci., USA, 96: 3906-3910 (1999)). In certain embodiments, a posttranscriptional regulatory element is a hepatitis B virus posttranscriptional regulatory element (HBVPRE) or a RNA transport element (RTE). In some embodiments, the WPRE or HBVPRE sequence is any of the WPRE or HBVPRE sequences disclosed in U.S. Pat. No. 6,136,597 or U.S. Pat. No. 6,287,814. In some embodiments, a WPRE sequence comprises or consists of:

(SEQ ID NO: 51)
aatcaacctc tggattacaa aatttgtgaa agattgactg atattcttaa ctatgttgct
ccttttacgc tgtgtggata tgctgcttta atgcctctgt atcatgctat tgcttcccgt
acggctttcg ttttctcctc cttgtataaa tcctggttgc tgtctcttta tgaggagttg
tggcccgttg tccgtcaacg tggcgtggtg tgctctgtgt ttgctgacgc aacccccact
ggctggggca ttgccaccac ctgtcaactc ctttctggga ctttcgcttt ccccctcccg
atcgccacgg cagaactcat cgccgcctgc cttgcccgct gctggacagg ggctaggttg
ctgggcactg ataattccgt ggtgttgtcg gggaagctga cgtcctttcc atggctgctc
gcctgtgttg ccaactggat cctgcgcggg acgtccttct gctacgtccc ttcggctctc
aatccagcgg acctcccttc ccgaggcctt ctgccggttc tgcggcctct cccgcgtctt
cgctttcggc ctccgacgag tcggatctcc ctttgggccg cctccccgcc tg.

In some embodiments, an rAAV vector comprises a poly A signal. PolyA signals may be derived from many suitable species, including, without limitation SV-40, human and bovine.

Another useful regulatory component that may be included in an rAAV vector is an internal ribosome entry site (IRES). An IRES sequence, or other suitable systems, may be used to produce more than one polypeptide from a single gene transcript. An IRES (or other suitable sequence) is used to produce a protein that contains more than one polypeptide chain or to express two different proteins from or within the same cell. An exemplary IRES is the poliovirus internal ribosome entry sequence. The IRES may be located 5′ or 3′ to the 21OH transgene in the rAAV vector. In other embodiments, an rAAV vector may comprise a nucleotide sequence encoding a 2A peptide that allows for expression of multiple polypeptides from a single promoter.

A recombinant “vector” or “rAAV vector” is derived from the wild type genome of a virus such as AAV by using molecular methods to remove the wild type genome from the virus, and replace it with a non-native nucleic acid, such as a heterologous polynucleotide sequence (e.g., a therapeutic gene expression cassette expressing 21OH). Typically, for AAV, one or both inverted terminal repeat (ITR) sequences of the wild-type AAV genome are retained in the AAV vector. An rAAV vector can be distinguished from a viral genome, because all (or a part) of the viral genome has been replaced with a non-native sequence with respect to the viral genomic nucleic acid. Incorporation of a non-native sequence such as a heterologous polynucleotide therefore defines the viral vector as a “recombinant” vector, which in the case of AAV can be referred to as an “rAAV vector.” An rAAV vector comprising a nucleic acid molecule encoding 21OH may also be referred to as a “CYP21 vector” or a “21OH vector.” As will be apparent from context, “vector” may refer to an isolated recombinant nucleotide sequence or an AAV particle or virion comprising a recombinant nucleotide sequence.

In some embodiments, an rAAV vector does not comprise any binding sites for miRNA (microRNA). In some embodiments, an rAAV vector comprises one, two, three, four, five or more binding sites for an miRNA that is expressed in cells where expression of the 21OH protein is not desired (i.e., detargeting). In some embodiments, an rAAV vector comprises one or more binding sites for miR-122. Binding of miR-122 to the 21OH-encoding sequence may reduce expression of this sequence in liver cells, where miR-122 is highly prevalent (Thakral and Ghoshal, Curr Gene Ther. 2015: 15(2): 142-150).

An rAAV nucleic acid sequence can be packaged into a virus (also referred to herein as a “particle” or “virion”) for subsequent infection (transformation) of a cell, ex vivo, in vitro or in vivo. Where a recombinant vector sequence is encapsidated or packaged into an AAV particle, the particle can be referred to as a “TAAV”. Such particles or virions will typically include proteins that encapsidate or package the vector genome. Particular examples include viral envelope proteins, and, in the case of AAV, capsid proteins.

The AAV components of the rAAV vectors and particles described herein may be selected from various AAV serotypes. In certain cases, an rAAV vector may comprise an AAV nucleic acid sequence from a rh10, AAV1, AAV2, AAV3, AAV4, AAV5, AAV6, AAV7, AAV8, AAV9, AAV10, AAV11, AAV12, or rh74 serotype. These AAV components may be readily isolated using various techniques from an AAV serotype. Such AAV may be isolated or obtained from academic, commercial, or public sources (e.g., the American Type Culture Collection, Manassas, VA). Alternatively, the AAV sequences may be obtained through synthetic or other suitable means by reference to published sequences such as are available in the literature or in databases such as, e.g., GenBank™, PubMed, or the like.

In certain embodiments, an rAAV vector or rAAV particle comprises an AAV nucleic acid sequence or AAV protein as disclosed in, e.g., U.S. Pat. No. 7,906,111 or U.S. Pat. No. 7,629,322, which are herein incorporated by reference in their entireties. In some embodiments, an rAAV vector or rAAV particle comprises an AAV nucleic acid sequence or AAV protein from AAV serotype AAV8 or its variants, as disclosed in, e.g., U.S. Pat. Nos. 7,282,199, 9,587,250 or 9,677,089, which are herein incorporated by reference in their entireties. In some embodiments, an rAAV vector or rAAV particle comprises an AAV nucleic acid sequence or AAV protein from AAV serotype AAV9 or its variants, as disclosed in, e.g., U.S. Pat. No. 7,198,951, incorporated herein by reference in its entirety. In some embodiments, an rAAV vector or rAAV particle comprises an AAV nucleic acid sequence or AAV protein from AAV serotype rh74 or its variants, as disclosed in, e.g., U.S. Pat. No. 9,840,719, incorporated herein by reference in its entirety.

In some aspects, an rAAV vector of the present disclosure comprises a nucleic acid molecule comprising at least one AAV ITR sequence. In certain embodiments, an rAAV vector comprises two ITR sequences, which ITR sequences may be of the same or different AAV serotypes. In certain cases, AAV ITRs may be selected from among any AAV serotype, including, without limitation, rh10, AAV1, AAV2, AAV3, AAV4, AAV5, AAV6, AAV7, AAV8, AAV9, AAV10, AAV11, AAV12, rh74, and other AAV serotypes. In some embodiments, an rAAV vector described herein comprises a genome comprising a sequence of one or two AAV2 ITRs.

The present disclosure further provides an rAAV particle comprising an rAAV vector described herein. Thus, in some aspects, the present disclosure relates to an rAAV particle comprising a nucleic acid molecule comprising at least one AAV ITR and a non-AAV nucleotide sequence (also referred to as a heterologous polynucleotide) encoding a 21OH protein, the non-AAV nucleotide sequence operably linked to a promoter. In some embodiments, an rAAV particle comprises at least one capsid protein selected from the group consisting of AAV serotype rh10, AAV1, AAV2, AAV3, AAV4, AAV5, AAV6, AAV7, AAV8, AAV9, AAV10, AAV11, AAV12, rh74, and other AAV serotypes.

In one embodiment, an rAAV vector is an rh10 AAV vector comprising human 21OH cDNA, an ITR from AAV2, and a CAG promoter consisting of the enhancer from the cytomegalovirus immediate-early gene, the promoter, splice donor and intron from the chicken β-actin gene, and the splice acceptor from the rabbit ß-globin gene. In a further embodiment, an rAAV vector comprises a nucleic acid sequence encoding a 21OH protein comprising SEQ ID NO: 1 and a CAG promoter comprising or consisting of SEQ ID NO:2. In one embodiment, an rAAV vector is an rh10 AAV vector comprising human 21OH cDNA, an ITR from AAV2, and a PGK promoter. In another embodiment, an rAAV vector comprises a nucleic acid sequence encoding a 21OH protein comprising SEQ ID NO: 1 and a PGK promoter comprising or consisting of SEQ ID NO:3.

In some embodiments, an rAAV vector or particle comprises AAV1 capsid and a nucleic acid molecule comprising human 21OH cDNA: a CAG, PGK, CBA or CB6 promoter: and, optionally, one or two AAV2 ITR sequences. In one embodiment, an rAAV vector or particle is the ssAAV1-PGK-CYP21HA vector containing a genome with AAV2 ITR sequences and encoding AAV1 capsid proteins. In some embodiments, an rAAV vector or particle is an AAV1-CAG-CYP21, AAV1-PGK-CYP21, AAV1-CBA-CYP21 or AAV1-CB6-CYP21 vector. In other embodiments, an rAAV vector or particle comprises AAV5 capsid and a nucleic acid molecule comprising human 21OH cDNA: a CAG, PGK, CBA or CB6 promoter; and, optionally, one or two AAV2 ITR sequences.

In one embodiment, an rAAV vector or particle is the ssAAV5-PGK-CYP21HA vector containing a genome with AAV2 ITR sequences and encoding AAV5 capsid proteins. In some embodiments, an rAAV vector or particle is an AAV5-CAG-CYP21, AAV5-PGK-CYP21, AAV5-CBA-CYP21 or AAV5-CB6-CYP21 vector. In yet other embodiments, an rAAV vector or particle is an AAV6 vector comprising human 21OH cDNA: a CAG, PGK, CBA or CB6 promoter; and, optionally, one or two AAV2 ITR sequences. In some embodiments, an rAAV vector or particle is an AAV6-CAG-CYP21, AAV6-PGK-CYP21, AAV6-CBA-CYP21 or AAV6-CB6-CYP21 virus. In further embodiments, an rAAV vector or particle comprises AAV8 capsid and a nucleic acid molecule comprising human 21OH cDNA: a CAG, PGK, CBA or CB6 promoter; and, optionally, one or two AAV2 ITR sequences. In some embodiments, an rAAV vector or particle is an AAV8-CAG-CYP21, AAV8-PGK-CYP21, AAV8-CBA-CYP21 or AAV8-CB6-CYP21 virus. In some embodiments, an rAAV vector or particle is an AAV9 vector comprising human 21OH cDNA: a CAG, PGK, CBA or CB6 promoter; and, optionally, one or two AAV2 ITR sequences. In some embodiments, an rAAV vector or particle is an AAV9-CAG-CYP21, AAV9-PGK-CYP21, AAV9-CBA-CYP21 or AAV9-CB6-CYP21 vector. In additional embodiments, an rAAV vector or particle comprises AAV10 capsid and a nucleic acid molecule comprising human 21OH cDNA: a CAG, PGK, CBA or CB6 promoter; and, optionally, one or two AAV2 ITR sequences. In some embodiments, an rAAV vector or particle is an AAV10-CAG-CYP21, AAV10-PGK-CYP21, AAV10-CBA-CYP21 or AAV10-CB6-CYP21 virus. In some embodiments, an rAAV vector or particle comprises rh10 AAV capsid and a nucleic acid molecule comprising human 21OH cDNA; a CAG, PGK, CBA or CB6 promoter; and, optionally, one or two AAV2 ITR sequences.

In one embodiment, an rAAV vector or particle is the AAVrh10-CAG-CYP21A2-HA virus containing a genome with AAV2 ITR sequences and encoding rh10 capsid proteins. In some embodiments, an rAAV vector or particle is an AAVrh10-CAG-CYP21, AAVrh10)-PGK-CYP21, AAVrh10-CBA-CYP21 or AAVrh10-CB6-CYP21 vector. In any of these embodiments, a promoter may comprise or consist of SEQ ID NO:2, SEQ ID NO:3, SEQ ID NO:48, or SEQ ID NO:49 or a nucleotide sequence at least 90%, at least 91%, at least 92%, at least 93%, at least 94%, at least 95%, at least 96%, at least 97%, at least 98%, or at least 99% identical to SEQ ID NO:2, SEQ ID NO:3, SEQ ID NO:48, or SEQ ID NO:49. In any of these embodiments, an rAAV vector may comprise a Kozak sequence. In some embodiments, a Kozak sequence may comprise or be transcribed to SEQ ID NO:44, SEQ ID NO:45, SEQ ID NO:46, or SEQ ID NO:47. In any of these embodiments, an rAAV vector may further comprise an HBVPRE sequence or a WPRE sequence (e.g., SEQ ID NO:51).

In some embodiments, an rAAV vector or particle is an AAV5 serotype vector or particle comprising a codon-optimized nucleotide sequence encoding human 21OH under the control of a CBA promoter, comprising a Kozak sequence and further with or without an miR-122 binding sequence. In one embodiment, an rAAV vector or particle is the AAV5-CBA-Kozak-COhCYP21-miR122 vector, which includes a CBA promoter, a Kozak sequence and the miR-122 miRNA binding site (Thakral and Ghoshal, Curr Gene Ther. 2015: 15(2): 142-150) for detargeting, and a codon-optimized (CO) human CYP21 transgene (SEQ ID NO:50). In some embodiments, an rAAV vector or particle is an AAV5-CBA-Kozak-hCYP21, AAV5-CBA-Kozak-hCYP21-miR122, or AAV5-CBA-Kozak-COhCYP21 vector. In any of these embodiments, the codon-optimized nucleotide sequence may comprise SEQ ID NO:50 and the Kozak sequences may comprise or be transcribed to SEQ ID NO:44, SEQ ID NO:45, SEQ ID NO:46, or SEQ ID NO:47. In any of these embodiments, an rAAV vector may further comprise an HBVPRE sequence or a WPRE sequence (e.g., SEQ ID NO:51).

In some embodiments, an rAAV vector or rAAV particle comprises a capsid (“Cap”) protein (e.g., including the vp1, vp2, vp3 and hypervariable regions), a viral replication (“Rep”) protein (e.g., rep 78, rep 68, rep 52, or rep 40), and/or a sequence encoding one or more such proteins. These AAV components may be readily utilized in a variety of vector systems and host cells. Such components may be used alone, in combination with other AAV serotype sequences or components, or in combination with elements from non-AAV viral sequences. As used herein, artificial AAV serotypes include, without limitation, AAV with a non-naturally occurring capsid protein. Such an artificial capsid may be generated by any suitable technique, using a selected AAV sequence (e.g., a fragment of a vp1 capsid protein) in combination with heterologous sequences which may be obtained from a different selected AAV serotype, non-contiguous portions of the same AAV serotype, from a non-AAV viral source, or from a non-viral source. An artificial AAV serotype may be, without limitation, a pseudotyped AAV, a chimeric AAV capsid, a recombinant AAV capsid, or a “humanized” AAV capsid. Pseudotyped vectors, wherein the capsid of one AAV is replaced with a heterologous capsid protein, are useful in the present disclosure. In one embodiment, the AAV is AAV2/5. In another embodiment, the AAV is AAV2/8. See, e.g., Mussolino et al., Gene Therapy, 18(7): 637-645 (2011): Rabinowitz et al., J Virol, 76(2): 791-801 (2002).

In some embodiments, vectors useful in compositions and methods described herein contain, at a minimum, sequences encoding a selected AAV serotype capsid, or a fragment thereof. In some embodiments, useful vectors contain, at a minimum, sequences encoding a selected AAV serotype rep protein, or a fragment thereof. Optionally, such vectors may contain both AAV cap and rep proteins. In vectors in which both AAV rep and cap are provided, the AAV rep and AAV cap sequences can both be of one serotype. Alternatively, vectors may be used in which the rep sequences are from one AAV serotype and the cap sequences are from a different AAV serotype. In one embodiment, the rep and cap sequences are expressed from separate sources (e.g., separate vectors, or a host cell and a vector). In another embodiment, these rep sequences are fused in frame to cap sequences of a different AAV serotype to form a chimeric AAV vector, such as AAV2/8 described in U.S. Pat. No. 7,282,199, incorporated herein by reference in its entirety.

A suitable rAAV can be generated by culturing a host cell which contains a nucleic acid sequence encoding an AAV serotype capsid protein, or fragment thereof, as defined herein: a functional rep gene: a nucleic acid molecule composed of, at a minimum, AAV inverted terminal repeats (ITRs) and a 21OH ((CYP21A2) nucleic acid sequence; and sufficient helper functions to permit packaging of the nucleic acid molecule into the AAV capsid protein. In some aspects, the present disclosure provides a host cell comprising an rAAV vector or an rAAV particle disclosed herein. The components required to be present in the host cell to package an rAAV vector in an AAV capsid may be provided to the host cell in trans. Alternatively, any one or more of the required components (e.g., vector, rep sequences, cap sequences, and/or helper functions) may be provided by a stable host cell which has been engineered to contain one or more of the required components using various methods. Most suitably, such a stable host cell will contain the required component(s) under the control of an inducible promoter. However, the required component(s) may be under the control of a constitutive promoter. Examples of suitable inducible and constitutive promoters are provided herein, in the discussion above of regulatory elements suitable for use with a non-AAV nucleotide sequence, i.e., 21OH. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contains the rep and/or cap proteins under the control of inducible promoters. Still other stable host cells may be generated.

The rAAV vector, rep sequences, cap sequences, and helper functions required for producing the rAAV of the present disclosure may be delivered to the packaging host cell in the form of any genetic element which transfers the sequences carried thereon. The selected genetic element may be delivered by any suitable method, including those described herein. The methods used to construct any embodiment of this disclosure, including generating rAAV particles, are known to those with skill in nucleic acid manipulation and include genetic engineering, recombinant engineering and synthetic techniques. See, e.g., Sambrook et al, Molecular Cloning: A Laboratory Manual, Cold Spring Harbor Press, Cold Spring Harbor, N.Y.; Fisher et al, J. Virol., 70:520-532 (1993); and U.S. Pat. No. 5,478,745, each of which are herein incorporated by reference in their entireties.

In one aspect, the present disclosure provides a method of producing an rAAV particle, the method comprising culturing a host cell containing: (a) a nucleic acid molecule comprising or consisting of an rAAV vector genome expressing 21OH as described herein: (b) a nucleic acid molecule encoding an AAV rep: (c) a nucleic acid molecule encoding at least one AAV capsid protein; and (d) sufficient helper functions for packaging the rAAV vector genome into the rAAV particle.

rAAV particles of the present disclosure may be purified by various methods. In one embodiment, an rAAV virus may be purified by anion exchange chromatography. See, e.g., U.S. Patent Publication No. 2018/0163183 A1. Further details regarding the construction and characterization of the AAV vectors and particles disclosed herein is described in International Patent Publication No. WO 2019/143803, which is incorporated herein by reference in its entirety for all purposes.

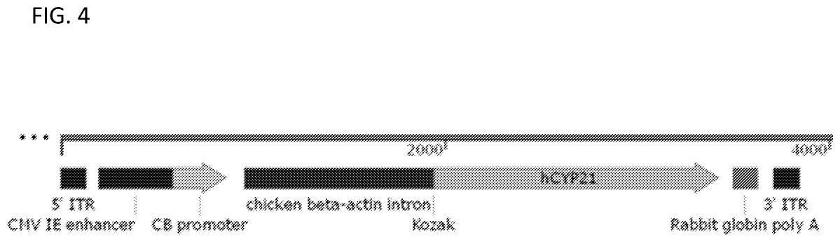

In some embodiments, the composition comprising the non-replicating, recombinant AAV serotype 5 (AAV5) vector containing an expression cassette for the human CYP21A2 transgene disclosed herein has the AAV-hCYP21-Opt vector design depicted in FIG. 4. In some embodiments, the sequence of the transgene that is packaged into rAAV5 particles is a single stranded DNA comprised of the following elements listed from 5′ to 3′: AAV 5′ inverted terminal repeat (ITR), the AAV packaging signal (Ψ), cytomegalovirus enhancer, chicken β-actin promoter including the canonical Kozak sequence with splice donor and rabbit β-globin intron with splice acceptor, the codon optimized human 21-hydroxylase (21-OH) cDNA of SEQ ID NO: 50, rabbit β-globin poly A signal, and the AAV 3′ inverted terminal repeat ITR.

In some embodiments, the expression cassette of the rAAV vector comprises or consists of the nucleic acid sequence of SEQ ID NO: 52. In some embodiments, the expression cassette of the rAAV vector comprises a 5′ ITR comprising the nucleic acid sequence of SEQ ID No: 53. In some embodiments, the expression cassette of the rAAV vector comprises a CMVIE Enhancer comprising the nucleic acid sequence of SEQ ID NO: 54. In some embodiments, the expression cassette of the rAAV vector comprises a CBA promoter comprising the nucleic acid sequence of SEQ ID NO: 55. In some embodiments, the expression cassette of the rAAV vector comprises an intron comprising the nucleic acid sequence of SEQ ID NO: 56. In some embodiments, the expression cassette of the rAAV vector comprises a codon optimized hCYP21 comprising the nucleic acid sequence of SEQ ID NO: 57. In some embodiments, the expression cassette of the rAAV vector comprises a beta-globin polyA comprising the nucleic acid sequence of SEQ ID NO: 58. In some embodiments, the expression cassette of the rAAV vector comprises a 3′ ITR comprising the nucleic acid sequence of SEQ ID NO: 59.

### Pharmaceutical Compositions

The rAAV vectors or particles of the present disclosure can be incorporated into pharmaceutical compositions suitable for administration. In one aspect, the present disclosure provides a pharmaceutical composition comprising an rAAV vector or an rAAV particle disclosed herein (e.g., an rAAV particle comprising a nucleic acid sequence encoding 21OH) and a pharmaceutically acceptable carrier, diluent or excipient. As used herein, the term “pharmaceutically acceptable” refers to molecular entities and compositions that do not generally produce allergic or other serious adverse reactions when administered using established routes. Molecular entities and compositions approved by a regulatory agency of the U.S. Federal or a U.S. state government or listed in the U.S. Pharmacopeia or other generally recognized pharmacopeia for use in animals, and more particularly in humans are considered to be “pharmaceutically acceptable”. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Some examples of such carriers or diluents include, but are not limited to, water, saline, buffered saline, Ringer's solutions, dextrose solution, 5% human serum albumin and other buffers, e.g., HEPES, to maintain pH at appropriate physiological levels. Except insofar as any media or agent is incompatible with the active compound, use thereof in the compositions is contemplated. Supplementary active compounds can also be incorporated into the compositions.

Throughout this description, “vg” may refer to “viral genomes” or “vector genomes.”

Examples of pharmaceutical compositions and delivery systems that may be used for administration of the rAAV disclosed herein can be found in Remington: The Science and Practice of Pharmacy (2003) 20^{th} ed., Mack Publishing Co., Easton, Pa.; Remington's Pharmaceutical Sciences (1990) 18^{th} ed., Mack Publishing Co., Easton, Pa.; The Merck Index (1996) 12^{th} ed., Merck Publishing Group, Whitehouse, N.J.; Pharmaceutical Principles of Solid Dosage Forms (1993), Technomic Publishing Co., Inc., Lancaster, Pa.: Ansel and Stoklosa, Pharmaceutical Calculations (2001) 11^{th} ed., Lippincott Williams & Wilkins, Baltimore, Md.; and Poznansky et al., Drug Delivery Systems (1980), R. L. Juliano, ed., Oxford, N.Y., pp. 253-315.

A pharmaceutical composition of the present disclosure may be formulated to be compatible with its intended route of administration. Examples of routes of administration include parenteral administration, e.g., intravenous administration, or injection. Injection may include direct injection into the adrenal gland via open surgery or laparoscopy or injection into an adrenal artery or adrenal vein via catheterization. Solutions or suspensions used for parenteral (e.g., intravenous or via injection) application can include the following components: a sterile diluent such as water for injection, saline solution, fixed oils, polyethylene glycols, glycerine, propylene glycol or other synthetic solvents; antibacterial agents such as benzyl alcohol or methyl parabens: antioxidants such as ascorbic acid or sodium bisulfate: chelating agents such as ethylenediaminetetraacetic acid (EDTA): buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, N.J.) or phosphate buffered saline (PBS). In all cases, the composition should be sterile and should be fluid to the extent that easy syringeability exists. The composition should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional ingredient from a previously sterile-filtered solution thereof.

For injection, a pharmaceutically acceptable carrier can be a liquid. Exemplary physiologically acceptable carriers include sterile, pyrogen-free water and sterile, pyrogen-free, phosphate buffered saline. A variety of such known carriers are provided in U.S. Pat. No. 7,629,322, which is herein incorporated by reference in its entirety. In one embodiment, the carrier is an isotonic sodium chloride solution. In another embodiment, the carrier is balanced salt solution. In one embodiment, the carrier includes TWEEN® (polysorbate). If the rAAV is to be stored long-term, it may be frozen in the presence of, e.g., glycerol or TWEEN® (polysorbate) 20.

It is especially advantageous to formulate parenteral compositions in dosage unit form for ease of administration and uniformity of dosage. Dosage unit form as used herein refers to physically discrete units suited as unitary dosages for the subject to be treated: each unit containing a predetermined quantity of active agent (e.g., rAAV) calculated to produce the desired therapeutic effect in association with the required pharmaceutical carrier. The specification for the dosage unit forms of the present disclosure are dictated by and directly dependent on the unique characteristics of the active agent (e.g., rAAV) and the particular therapeutic effect to be achieved, and the limitations inherent in the art of compounding such an active agent (e.g., rAAV) for the treatment of individuals. Unit dosage forms may be within, for example, ampules and vials, which may include a liquid composition, or a composition in a freeze-dried or lyophilized state: a sterile liquid carrier, for example, can be added prior to administration or delivery in vivo. Individual unit dosage forms can be included in multi-dose kits or containers. Recombinant vector (e.g., rAAV) sequences, plasmids, vector genomes, recombinant virus particles (e.g., rAAV), and pharmaceutical compositions thereof can be packaged in single or multiple unit dosage form for ease of administration and uniformity of dosage.

A pharmaceutical composition comprising an rAAV vector or an rAAV particle comprising a nucleic acid sequence encoding 21OH can be included in a container, pack, or dispenser together with instructions for administration.

In some embodiments, the composition is a single-dose, preservative-free, sterile, opalescent, colorless solution, IV injection, of the non-replicating, recombinant AAV5 vector disclosed herein at a target concentration of 5.0×10¹³ vg/mL (3.0×10¹³ to 7.0×10¹³ vg/mL). In some embodiments, the composition contains 10 mM Sodium Phosphate, 180 mM Sodium Chloride, 0.005% w/v poloxamer 188 and Water for Injection. In some embodiments, the composition is filled into 5 mL Crystal Zenith® (CZ, cyclic olefin polymer) vials with a nominal fill volume of 2.5 mL and stored at ≤−60° C.

In an aspect, the present disclosure provides a kit comprising an rAAV vector, or a particle comprising the same, where the rAAV vector comprises a nucleic acid molecule comprising at least one AAV inverted terminal repeat (ITR) and a non-AAV nucleotide sequence encoding a 21-hydroxylase (21OH) protein, where the non-AAV nucleotide sequence is operably linked to a promoter; and where the therapeutically effective amount is in the range of about 10¹² vg/kg to about 10¹⁴ vg/kg. In some embodiments, the therapeutically effective amount is about 1.5×10¹³ vg/kg. In some embodiments, the therapeutically effective amount is about 3×10¹³ vg/kg. In some embodiments, the therapeutically effective amount is about 6×10¹³ vg/kg. In some embodiments, the kit further comprises instructions for administering the rAAV vector to a subject. In some embodiments, the kit further comprises instructions for administering the rAAV vector intravenously, by direct injection into the adrenal gland via open surgery or laparoscopy, or by injection into an adrenal artery or adrenal vein via catheterization to a subject.

In another aspect, the present disclosure provides a unit dose comprising an rAAV vector, or a particle comprising the same, where the rAAV vector comprises a nucleic acid molecule comprising at least one AAV inverted terminal repeat (ITR) and a non-AAV nucleotide sequence encoding a 21-hydroxylase (21OH) protein, where the non-AAV nucleotide sequence is operably linked to a promoter; and where the therapeutically effective amount is in the range of about 10¹² vg/kg to about 10¹⁴ vg/kg. In some embodiments, the therapeutically effective amount is about 1.5×10¹³ vg/kg. In some embodiments, the therapeutically effective amount is about 3×10¹³ vg/kg. In some embodiments, the therapeutically effective amount is about 6×10¹³ vg/kg. In some embodiments, the unit dose comprises a liquid formulation. In some embodiments, the unit dose is configured for administration to a subject intravenously, by direct injection into the adrenal gland via open surgery or laparoscopy, or by injection into an adrenal artery or adrenal vein via catheterization.

In some embodiments of the above aspects, the 21OH protein is human 21OH protein. In some embodiments of the above aspects, the non-AAV nucleotide sequence encoding a 21OH protein comprises or consists of the human 21OH (CYP21A2) cDNA. In some embodiments of the above aspects, the non-AAV nucleotide sequence encoding a 21OH protein comprises or consists of a codon-optimized nucleotide sequence. In some embodiments of the above aspects, the non-AAV nucleotide sequence encoding a 21OH protein comprises or consists of SEQ ID NO: 50. In some embodiments of the above aspects, the non-AAV nucleotide sequence encoding a 21OH protein encodes the amino acid sequence of SEQ ID NO: 1 or an amino acid sequence at least 95%, at least 96%, at least 97%, at least 98% or at least 99% identical to SEQ ID NO: 1. In some embodiments of the above aspects, the promoter directs expression of the 21OH protein in a host cell. In some embodiments of the above aspects, the host cell is an adrenal gland cell or an adrenal cortex cell. In some embodiments of the above aspects, the promoter is a cytomegalovirus/β-actin hybrid promoter, PGK promoter, or a promoter specific for expression in an adrenal cortex cell. In some embodiments of the above aspects, the cytomegalovirus/β-actin hybrid promoter is a CAG, CB6, or CBA promoter. In some embodiments of the above aspects, the promoter comprises or consists of the nucleotide sequence of SEQ ID NO: 2, SEQ ID NO: 3, SEQ ID NO:48, or SEQ ID NO:49. In some embodiments of the above aspects, the ITR is an AAV1, AAV2, AAV3, AAV4, AAV5, AAV6, AAV7, AAV8, AAV9, AAV10, AAV11, AAV12, rh10, or rh74 serotype ITR. In some embodiments of the above aspects, the rAAV is an AAV1, AAV2, AAV3, AAV4, AAV5, AAV6, AAV7, AAV8, AAV9, AAV10, AAV11, AAV12, rh10, or rh74 serotype. In some embodiments of the above aspects, the rAAV is an AAV5 serotype. In some embodiments of the above aspects, wherein the nucleic acid molecule further comprises a Kozak sequence. In some embodiments of the above aspects, the nucleic acid molecule further comprises an miR-122 binding site.

### Methods of Using Recombinant AAV Vectors and Particles

The present disclosure provides methods involving the use of AAV (e.g., recombinant AAV) vectors, such as in the treatment of a subject in need. The methods provided herein comprise, e.g., administering to a subject a therapeutically effective amount of any one of the rAAVs or compositions disclosed herein.

In certain embodiments, a subject is a human, a non-human primate, a pig, a horse, a cow; a dog, a cat, a rabbit, a guinea pig, a hamster, a mouse, or a rat. In particular embodiments, the subject is human. A human subject may be a human female or a human male. In some embodiments, the subject is a human that has previously undergone a hormone therapy program. In some embodiments, a subject is a human infant. In certain cases, a subject is a human infant about 1 month old, about 2 months old, about 3 months old, about 4 months old, about 5 months old, about 6 months old, about 7 months old, about 8 months old, about 9 months old, about 10 months old, about 11 months old, or about 1 year old. In some embodiments, a subject is a human infant less than 3 months old, less than 6 months old, less than 9 months old, less than 1 year old, or less than 18 months old.

As used herein, the term “patient in need” or “subject in need” refers to a patient or subject at risk of, or suffering from, a disease, disorder or condition that is amenable to treatment or amelioration with an rAAV comprising a nucleic acid sequence encoding 21OH or a composition comprising such an rAAV provided herein. A patient or subject in need may, for instance, be a patient or subject diagnosed with a disease associated with the malfunction of 21OH, such as 21OHD, such as CAH. A subject may have a mutation or a malfunction in a 21OH gene or protein. “Subject” and “patient” are used interchangeably herein.

As used herein, the term “effective amount” or “therapeutically effective amount” refers to the amount of a pharmaceutical agent, e.g., an rAAV, which is sufficient to reduce or ameliorate the severity and/or duration of a disorder, e.g., 21OHD, or one or more symptoms thereof, prevent the advancement of a disorder, cause regression of a disorder, prevent the recurrence, development, onset or progression of one or more symptoms associated with a disorder, detect a disorder, or enhance or improve the prophylactic or therapeutic effect(s) of another therapy (e.g., prophylactic or therapeutic agent). The effective amount of an rAAV may, for example, increase the expression of 21OH, and/or relieve to some extent one or more of the symptoms associated with 21OHD.

The disclosure provides methods and uses of the rAAV, comprising a nucleic acid molecule encoding 21OH as described herein for providing a therapeutic benefit to a subject with a disorder or a disease characterized by a deficiency or malfunction of 21OH. In some aspects, a method comprises administering to a subject in need thereof, a therapeutically effective amount of an rAAV or a composition described herein, thereby treating said disorder or said disease characterized by a deficiency or malfunction of 21OH in the subject.

In some cases, the present disclosure provides a method of expressing 21OH in a subject in need thereof, comprising administering to the subject a therapeutically effective amount of an rAAV particle described herein or a pharmaceutical composition described herein, thereby expressing 21OH in the subject. In certain embodiments, the present disclosure provides a method of increasing the expression of 21OH in a subject in need thereof, comprising administering to the subject a therapeutically effective amount of an rAAV particle described herein or pharmaceutical composition comprising said particle, thereby increasing expression of 21OH in the subject. In some embodiments, the methods disclosed herein result in the expression of 21OH or increases the expression of 21OH in the subject's adrenal gland (e.g., adrenal cortex or adrenal medulla), liver or ovary. In certain embodiments, the methods disclosed herein result in the expression of 21OH or increases the expression of 21OH in the subject's adrenal stem cells (e.g., adrenocortical stem cells) or adrenal progenitor cells. In certain embodiments, the methods disclosed herein result in the expression of 21OH or increases the expression of 21OH in the subject's liver or ovary.

The disclosure provides methods of treating a subject with 21-hydroxylase deficiency (21OHD), comprising administering to the subject a therapeutically effective amount of an rAAV particle described herein or pharmaceutical composition comprising said particle, thereby treating 21OHD in the subject. The disclosure provides methods of increasing the level of cortisol and/or aldosterone in a subject in need thereof, comprising: administering to the subject a therapeutically effective amount of an rAAV particle or a composition disclosed herein, thereby increasing the level of cortisol and/or aldosterone in the subject. The disclosure provides methods of decreasing the level of progesterone, 17-OHP, renin and/or androstenedione (A4) in a subject in need thereof, comprising: administering to the subject a therapeutically effective amount of an rAAV particle or a composition disclosed herein, thereby decreasing the level of progesterone, 17-OHP, renin and/or androstenedione (A4) in the subject.

The disclosure further provides methods of treating, reducing, improving, slowing the progression of or preventing a symptom of 21OHD (or congenital adrenal hyperplasia, CAH) in a subject having 21OHD (or CAH), the method comprising administering to the subject a therapeutically effective amount of an rAAV particle described herein or pharmaceutical composition comprising said particle, thereby treating, reducing, improving, slowing the progression of or preventing a symptom of 21OHD in a subject. Non-limiting examples of symptoms of 21OHD (or CAH) include genital and muscle mass virilization, salt wasting and dehydration in infancy, impaired sexuality (classical forms) and hirsutism, acne, and decreased fertility (classical and non-classical forms). In a male patient, non-limiting examples of symptoms of 21OHD (or CAH) include short stature and premature virilization. The Prader classification system is used to measure the degree of virilization of the genitalia of the human body. In some embodiments, a subject (e.g., female subject) treated by the methods of the present disclosure has ambiguous genitalia by Prader staging (e.g., the subject is affected with Prader stage IV or V form of 21OHD (or CAH).

In some embodiments, the subject is in need of expression of 21OH. In some embodiments, the subject has a 21OH deficiency (21OHD). In some embodiments, a subject (e.g., female subject) has ambiguous genitalia by Prader staging (e.g., the subject is affected with Prader stage IV or V form of 21OHD (or CAH). In some embodiments, the subject has congenital adrenal hyperplasia (CAH). In some embodiments, the CAH is classical CAH. In some embodiments, the subject is in need of cortisol and/or aldosterone. In some embodiments, the subject has a cortisol deficiency and/or an aldosterone deficiency. In some embodiments, the subject has an excess of progesterone, 17-OHP, renin, and/or androstenedione (A4). In some embodiments, the subject has an excess of progesterone, 17-OHP, renin, and/or androstenedione (A4) in the blood and/or urine.

In some embodiments, the subject has more than about 5 to about 10 times ULN (upper limit of normal) of 17-OHP. In some embodiments, the subject has androstenedione (A4) value above ULN. In some embodiments, the subject has an 11-oxo or 11-keto sterol (e.g., 11-ketotestosterone) value above ULN. In some embodiments, the subject is on a stable regimen of oral hydrocortisone as the only glucocorticoid maintenance therapy at a total daily dose of about 30 milligrams per day (mg/day) hydrocortisone. In some embodiments, the subject is on a stable regimen of oral hydrocortisone as the only glucocorticoid maintenance therapy at a total daily dose of about 10 mg/day to about 50 mg/day hydrocortisone.

In some embodiments, the methods further comprise selecting a subject with 21OHD before the administering step. In some embodiments, the subject may be screened and identified or diagnosed as having 21OHD (e.g., by genetic or physiological testing) even though the subject does not have one or more symptoms of the disease. In other embodiments, a subject has one or more symptoms of 21OHD. In certain embodiments, a subject has a mutation in a CYP21A2 gene. In one embodiment, a subject has a loss-of-function mutation in a CYP21A2 gene. In certain embodiments, a subject has a defect in a CYP21A2 gene caused by gene conversion or recombination between the functional CYP21A2 gene and the closely linked, non-functional CYP21A1P pseudogene.

In some embodiments, there is a higher level of cortisol and/or aldosterone in the subject after administration of the rAAVs, or compositions disclosed herein, as compared to before administration of the rAAVs or compositions. In some embodiments, there is a higher level of cortisol and/or aldosterone in the subject after administration of the rAAVs or compositions disclosed herein, as compared to a control subject having 21-hydroxylase deficiency (21OHD), who has not been administered the rAAVs or compositions. In some embodiments, there is a lower level of progesterone, 17-OHP, renin and/or androstenedione (A4) in the subject after administration of the rAAVs or compositions disclosed herein, as compared to before administration of the rAAVs or compositions. In some embodiments, there is a lower level of progesterone, 17-OHP, renin and/or androstenedione (A4) in the subject after administration of the rAAVs or compositions disclosed herein, as compared to a control subject having 21-hydroxy lase deficiency (21OHD), who has not been administered the rAAVs or compositions.

In some embodiments of the methods disclosed herein, a hepatotoxic agent (e.g., ketamine, halogenated anesthetics) is not administered to the subject in combination with the rAAV or composition disclosed herein. In some embodiments of the methods disclosed herein, the hepatotoxic agent is not administered for at least 12 weeks after receiving an rAAV or composition disclosed herein. In some embodiments of the methods disclosed herein, acetaminophen (and other similar medications) is not administered to the subject in combination with the rAAV or composition disclosed herein. In some embodiments of the methods disclosed herein, substances that inhibit or induce tacrolimus metabolism via the CYP3A or CYP3A4 pathway, including grapefruit juice is not administered to the subject in combination with the rAAV or composition disclosed herein.

### Modes of Administration

In any of the treatment methods described herein, an rAAV particle comprising a nucleic acid molecule encoding 21OH or a pharmaceutical composition comprising said particle may be administered to a subject by any means of introducing said rAAV particle into the adrenal cortex vasculature or the adrenal cortex itself. In some embodiments, an rAAV particle comprising a nucleic acid molecule encoding 21OH or a pharmaceutical composition comprising said particle may be administered to a subject systemically. In some embodiments, an rAAV particle comprising a nucleic acid molecule encoding 21OH or a pharmaceutical composition comprising said particle may be administered to a subject intravenously: by direct injection into the adrenal gland via open surgery or laparoscopy: by injection into an adrenal artery or adrenal vein via catheterization. In some embodiments, the direct injection into the adrenal gland is direct injection into the adrenal cortex.

In some embodiments, an rAAV particle comprising a nucleic acid molecule encoding 21OH as described herein or a pharmaceutical composition comprising said particle may be used to treat a subject suffering from congenital adrenal hyperplasia (CAH). Deficiency of 21OH often leads to CAH, a family of inherited disorders affecting the adrenal glands. CAH may be present in a subject in a severe or a mild form. The severe form, called classical CAH or classic CAH, is usually detected in the newborn period or in early childhood. The milder form, called non-classical CAH (NCAH or NCCAH) or late-onset CAH, may cause symptoms at any time from infancy through adulthood (see, e.g., Kurtoğlu et al., J Clin Res Pediatr Endocrinol, 9(1): 1-7 (2017)). The rAAV of the present disclosure may be used to treat a subject with classical CAH or non-classical CAH. Subjects with classical CAH may experience fetal masculinization of external genitals, low or absent glucocorticoid and mineralocorticoid production, and/or production of a large excess of androgens. Subjects with non-classical CAH may experience increased production of androgens without fetal masculinization and without cortisol and aldosterone deficits.

Cortisol is a steroid produced by the adrenal glands. Cortisol is used in the body to respond to physical and emotional stress, and maintain adequate energy supply and blood sugar levels. The adrenal glands are controlled by the pituitary gland, a small pea-sized gland at the base of the brain. In healthy individuals, the pituitary gland releases adrenocorticotropic hormone (ACTH) when there is insufficient cortisol present in the bloodstream. ACTH stimulates the adrenals to produce more cortisol. However, those with CAH have insufficient amounts of 21OH, which is needed to convert the precursor 17-hydroxyprogesterone (17-OHP) into cortisol. As a result, the pituitary gland continues to sense the need for cortisol and releases more ACTH. This leads to an overabundance of 17-OHP, which is then converted in the adrenals into excess androgens (masculinizing steroid hormones). As such, a subject may be diagnosed with CAH by determining increased circulating levels of the affected steroid hormones. Neonatal screening for 21OHD is typically accomplished using a 17-OHP measurement. Additionally, a subject with CAH may be monitored by tracking circulating levels of 17-OHP. Thus, in some embodiments, the circulating levels of the affected steroid hormones may be measured in a subject with CAH before, during and/or after treatment with an rAAV particle comprising a nucleic acid molecule encoding 21OH as described herein or a pharmaceutical composition comprising said particle. The circulating levels of 17-OHP in a subject may be used for diagnosis of a subject and to inform a decision about whether to begin or to continue treatment of the subject with an rAAV particle or pharmaceutical composition described herein.

In some embodiments, the methods disclosed herein have one or more of the following effects: (1) decrease or eliminate reliance on exogenous glucocorticoids (GC) by restoring physiologic endogenous cortisol biosynthesis, thereby reducing sequelae of supraphysiologic GC therapy: (2) reduce the hyperandrogenism associated with 21-OHD without increases in exogenous GC dosing, thereby also reducing the sequelae of hyperandrogenism: (3) reduce the risk of life-threatening adrenal crisis related by restoring endogenous cortisol and/or aldosterone biosynthesis; and (4) reduce patient burden and non-compliance related to daily dosing of supraphysiologic GC and/or mineralocorticoid (MC) replacement therapy. Thus, in some embodiments, the methods disclosed herein alleviate the adrenal hormonal deficits in CAH and, thereby, help ameliorate the underlying pathobiology of the disease.

The present disclosure further contemplates a use of a pharmaceutical agent (e.g., an rAAV or a pharmaceutical composition comprising an rAAV) described herein in the manufacture of a medicament for treating a disorder or a disease characterized by a malfunction or a deficiency of 21OH in a subject. The present disclosure also includes a use of a pharmaceutical agent (e.g., an rAAV or a pharmaceutical composition comprising an rAAV) described herein for treating a disorder or a disease characterized by a malfunction or a deficiency of 21OH in a subject.

### Dosage

The composition may be delivered in a volume of from about 50 microliters (μL) to about 1 milliliter (mL), including all numbers within the range, depending on the size of the area to be treated, the viral titer used, the route of administration, and the desired effect of the method. In one embodiment, the volume is about 50 μL. In another embodiment, the volume is about 70 μL. In another embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 250 μL. In another embodiment, the volume is about 300 μL. In another embodiment, the volume is about 450 μL. In another embodiment, the volume is about 500 μL. In another embodiment, the volume is about 600 μL. In another embodiment, the volume is about 750 μL. In another embodiment, the volume is about 850 μL. In another embodiment, the volume is about 1000 μL.

In some embodiments, an effective concentration of an rAAV carrying a nucleic acid sequence encoding the particular transgene (e.g., 21OH) under the control of a promoter sequence ranges between about 10⁸ and about 10¹³ vector genomes per milliliter (vg/mL). For example, the rAAV infectious units may be measured as described in Mclaughlin et al, J. Virol., 62:1963 (1988). In some embodiments, the concentration is from about 1.5×10⁹ vg/ml to about 1.5×10¹² vg/mL. In some embodiments, the concentration is from about 1.5×10⁹ vg/mL to about 1.5×10¹¹ vg/mL. In one embodiment, the effective concentration is about 1.5×10¹⁰ vg/mL. In another embodiment, the effective concentration is about 1.5×10¹¹ vg/mL. In another embodiment, the effective concentration is about 2.8×10¹¹ vg/mL. In yet another embodiment, the effective concentration is about 1.5×10¹² vg/mL. In a further embodiment, the effective concentration is about 1.5×10¹³ vg/mL. In some embodiments, the concentration is in the range of about 10¹³ vg/mL to about 7.0×10¹³ vg/mL, for example, about 2×10¹³ vg/mL, about 3×10¹³ vg/mL, about 4×10¹³ vg/mL, about 5×10¹³ vg/mL, about 6×10¹³ vg/mL, about 7×10¹³ vg/mL, including all values and subranges that lie therebetween. In some embodiments, the concentration is about 5×10¹³ vg/mL.

In some embodiments, the lowest effective concentration of virus is utilized in order to reduce the risk of undesirable effects, such as toxicity or adverse immune response. Still other dosages in these ranges may be selected by the attending physician, taking into account the physical state of the subject (e.g., human) being treated, the age of the subject, the particular 21OH deficiency disorder and the degree to which the disorder, if progressive, has developed.

In some embodiments, rAAV vectors or rAAV particles comprising a nucleic acid sequence encoding 21OH are administered to a subject at a dose ranging from about 10¹¹ to about 10¹⁴ vg/kg body weight of the subject, such as from about 10¹² vg/kg to about 10¹⁴ vg/kg body weight of the subject. In some embodiments, rAAV vectors or rAAV particles comprising a nucleic acid sequence encoding 21OH are administered to a subject at a dose of about 1.5×10¹³ vg/kg, 3×10¹³ vg/kg, or 6×10¹³ vg/kg.

In some embodiments, the therapeutically effective amount is in the range of about 10¹² vg/kg to about 10¹⁴ vg/kg, for example, about 5×10¹² vg/kg, about 10¹³ vg/kg, or about 5×10¹³ vg/kg, including all values and subranges that lie therebetween. In some embodiments, the therapeutically effective amount is in the range of about 10¹³ vg/kg to about 10¹⁴ vg/kg, for example, about 1.5×10¹³ vg/kg, 2×10¹³ vg/kg, 2.5×10¹³ vg/kg, 3×10¹³ vg/kg, 3.5×10¹³ vg/kg, 4×10¹³ vg/kg, 4.5×10¹³ vg/kg, 5×10¹³ vg/kg, 5.5×10¹³ vg/kg, 6×10¹³ vg/kg, 6.5×10¹³ vg/kg, 7×10¹³ vg/kg, 7.5×10¹³ vg/kg, 8×10¹³ vg/kg, 8.5×10¹³ vg/kg, 9×10¹³ vg/kg, or 9.5×10¹³ vg/kg, including all values and subranges that lie therebetween. In some embodiments, the therapeutically effective amount is about 1.5×10¹³ vg/kg. In some embodiments, the therapeutically effective amount is about 3×10¹³ vg/kg. In some embodiments, the therapeutically effective amount is about 6×10¹³ vg/kg.

In some embodiments, the subject is administered a single dose of the rAAV vectors, rAAV particles, or compositions disclosed herein. In some embodiments, the subject is administered more than one dose of the rAAV vectors, rAAV particles, or compositions disclosed herein, for example, two, three, four, or five doses.

The compositions disclosed herein may be administered to the subject at any frequency. In some embodiments, the composition may be administered to the subject once a day or more than once a day. In some embodiments, the composition may be administered to the subject 2, 3, 4, 5, 6, 7, 8, 9, or 10 times a day. In some embodiments, the composition may be administered to the subject every day, every alternate, every third day, every fourth day, every fifth day, or every sixth day. In some embodiments, the composition may be administered to the subject weekly, bi-weekly or every three weeks. In some embodiments, the composition may be administered to the subject every 1, 2, 3, 4, 5, 6, 7, 8, 9, 10, or 11 months. In some embodiments, the composition may be administered to the subject every year. In some embodiments, the composition may be administered to the subject every 1, 2, 3, 4, 5, 10, 15, or 20 years. In a particular embodiment, the composition may be administered to the subject a single time during the subject's lifetime.

Combination with Immunosuppressants

In some embodiments, any one of the methods disclosed herein further comprise administering a therapeutically effective amount of an immunosuppressant. In some embodiments, the methods comprise administering to the subject a therapeutically effective amount of an rAAV or a composition disclosed herein, and a therapeutically effective amount of an immunosuppressant. The disclosure provides methods of treating administering to the subject a therapeutically effective amount of an rAAV or a composition disclosed herein, and a therapeutically effective amount of an immunosuppressant.

In some embodiments, the immunosuppressant is administered before, concurrently with, and/or after the administration of the rAAV vector. In some embodiments, the immunosuppressant is administered before the administration of the rAAV vector. In some embodiments, the immunosuppressant is administered at least 12 hours before the administration of the rAAV vector. In some embodiments, the immunosuppressant is administered about 2 days before the administration of the rAAV vector.

In some embodiments, the immunosuppressant is a non-glucocorticoid immunosuppressant. In some embodiments, the immunosuppressant is an inhibitor of calcineurin. In some embodiments, the immunosuppressant is cyclosporin, tacrolimus, sirolimus, everolimus, zotarolimus, or any combination thereof. In some embodiments, the immunosuppressant is tacrolimus. Other non-limiting examples of immunosuppressants include alkylating agents such as nitrogen mustards (cyclophosphamide), nitrosoureas, platinum compounds, folic acid analogues, such as methotrexate, purine analogues, such as azathioprine and mercaptopurine, pyrimidine analogues, such as fluorouracil, protein synthesis inhibitors, cytotoxic antibodies such as dactinomycin, anthracyclines, mitomycin C, bleomycin, mithramycin, polyclonal antibodies inhibiting T lymphocytes, IL-2 receptor-directed monoclonal antibodies such as basiliximab or daclizumab, anti-CD3 monoclonal antibodies, such as muromonab, opioids, TNF-alpha binding proteins such as infliximab, etanercept, or adalimumab, mycophenolate, fingolimod and myriocin.

In some embodiments, the immunosuppressant is administered orally. In some embodiments, the therapeutically effective amount of the immunosuppressant is in the range of about 0.005 milligrams per kilogram (mg/kg) to about 0.1 mg/kg, for example, about 0.01 mg/kg, about 0.015, about 0.02, about 0.025, about 0.03, about 0.035, about 0.04, about 0.045, about 0.05, about 0.06, about 0.07, about 0.08, about 0.09, or about 0.1 mg/kg, including all values and subranges that lie therebetween. In some embodiments, the therapeutically effective amount of the immunosuppressant is in the range of about 0.01 mg/kg to about 0.05 mg/kg. In some embodiments, the therapeutically effective amount of the immunosuppressant is about 0.025 mg/kg. In some embodiments, the therapeutically effective amount of the immunosuppressant is administered twice daily.

In some embodiments, the methods comprise administering a therapeutically effective amount of a steroid to the subject. In some embodiments, the steroid is a mineralocorticoid. In some embodiments, the steroid is a glucocorticoid. In some embodiments, the steroid is hydrocortisone. In some embodiments, the steroid is administered before, concurrently with, and/or after administration of the rAAV vector. In some embodiments, the therapeutically effective amount of the steroid administered to the subject before the administration of the rAAV vector is higher than the therapeutically effective amount of the steroid administered to the subject after the administration of the rAAV.

### Examples

### Example 1: Rescue of Congenital Adrenal Hyperplasia (CAH) symptoms in mice

Construction of AAV vectors encoding 21-hydroxylase (21OH) protein was done as described in International Patent Publication No. WO 2019/143803, which is incorporated herein by reference in its entirety for all purposes.

The H-2^{aw18} (Cyp21^{-/-}) mouse is the only animal model of human CAH due to 21-OHD that mimics the key pathophysiologic features of the clinical condition of CAH, i.e., presenting with failure to thrive that leads to postnatal morbidity due to glucocorticoid (GC) and mineralocorticoid (MC) deficiency. An AAV serotype 5 (AAV5) vector containing a codon-optimized human CYP21A2 transgene of SEQ ID NO: 50, under control of the CBA promoter (also referred to herein as “BBP-631”) was administered to 15-week old Cyp21^{-/-} mice and disease phenotypes compared with untreated Cyp21^{-/-} mice for at least 10 weeks. Vector genome copies (VGC) of human CYP21A2 transgene and mRNA normalized to mouse GAPDH were measured in adrenals and other organs. Adrenal 21-OH protein expression was evaluated using customized nano-LCMSMS proteomics to detect a peptide specific to human 21-OH, normalized to GAPDH and endogenous murine 21-OH peptides.

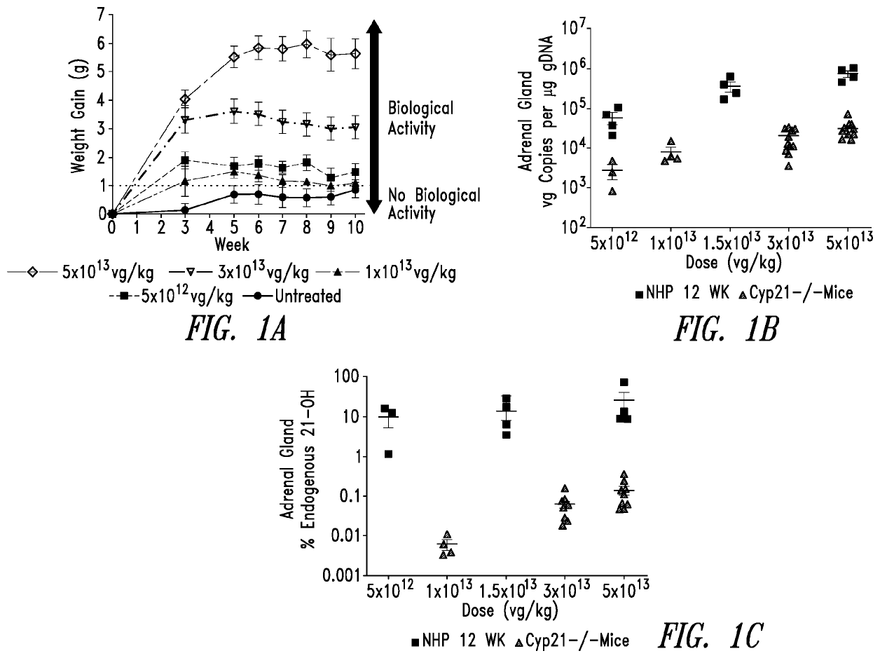

The results showed that the administration of the AAV5 comprising a functional copy of the human CYP21A2 gene led to early and sustained disease rescue of the Cyp21^{-/-} mice accompanied by the following: (i) reduction of urinary progesterone levels over all 10 weeks, consistent with restoration of 21-hydroxylation of progesterone: (ii) reduction of renin expression in the kidney, suggesting improvement in MC function; and (iii) dose-dependent detection of vector genomes, human CYP21A2 mRNA, and human 21-OH protein in the adrenal gland. Treated mice demonstrated a dose dependent weight gain that correlated with adrenal VGC, transgene mRNA level, and human 21-OH protein expression. The dose-dependent weight gain and the dose-dependent detection of vector genomes in the adrenal gland of mice are shown in FIG. 1A and FIG. 1B, respectively.

In conclusion, gene therapy of Cyp21^{-/-} mice with AAV serotype 5 (AAV5) vector containing a codon-optimized human CYP21A2 transgene under control of the CBA promoter was robust and dose-dependent at the molecular level and for the correction of disease phenotypes.

In WT mice, a GLP-compliant toxicology study evaluated safety (clinical observations, pathology, hematology, clinical chemistry, histopathology, and immune response) and biodistribution at Weeks 4, 12 and 24. Vector genome and transgene expression were detected in adrenal gland and other tissues. No test-article related toxicity was observed in WT C57BL/6 mice at any study timepoint following a single IV administration of BBP-631 at any dose tested. This study demonstrates that BBP-631 is well-tolerated with a no-observed-adverse-effect level (NOAEL) of 3.0×10¹⁴ vg/kg, and has durable transgene transfer and expression in adrenal glands.

### Example 2: Expression of 21-OH in Non-Human Primates (NHPs)

Persistent, dose-dependent expression of human 21-OH protein in adrenal glands of NHPs administered 1 dose of the AAV vector encoding 21-hydroxylase (21OH) protein (BBP-631) is shown in FIG. 1B and FIG. 1C. The amount of human 21-OH produced, expressed as percentage of endogenous 21-OH in NHPs, suggests the potential for clinically meaningful disease impact in people with classical CAH (FIG. 1C). Identification of biologically active doses in mice and NHPs support the starting dose in the Phase 1/2 clinical study with the disclosed AAV serotype 5 (AAV5) vector containing a codon-optimized human CYP21A2 transgene under control of the CB6 promoter.

A biodistribution and safety study was performed in 2-2.5-year old NHPs (cynomolgus macaques). Following IV administration of AAV5 comprising a functional copy of the human CYP21A2 gene, no adverse safety signals were seen for clinical observations, body or organ weights, urinalysis, hematology, serum chemistry, gross pathology, histopathology (including thoracic spinal cord), immunohistochemistry (no CD4-lymphocyte infiltrates), or anti-drug responses (cell-mediated immune responses to AAV5 capsid or human 21-OH) across all doses. Dose-dependent viral genome copies (VGC) and stable mRNA transcript expression were detected in adrenals through Week 24. Proteomic methods showed a dose-dependent and persistent expression of human 21-OH protein up to 9 to 24% of endogenous 21-OH levels. Overall, the results indicate that delivery of AAV5 comprising a functional copy of the human CYP21A2 gene is well tolerated with persistent expression in adrenals of NHPs for a duration of at least 24 weeks.

### Example 3: Phase 1/2 Clinical Study

### Overview

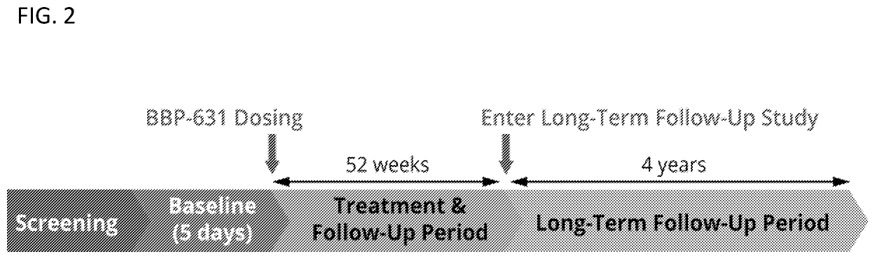

A Phase 1/2, first in human, open-label, dose-escalation study was designed to evaluate the safety, tolerability, and efficacy of an AAV serotype 5 (AAV5) vector containing a codon-optimized human CYP21A2 transgene under control of the CB6 promoter (also referred to herein as “BBP-631”) administered to up to 25 adult participants diagnosed with classical congenital adrenal hyperplasia (CAH) (simple virilizing or salt-wasting, Group 1) or with classical salt-wasting CAH (Group 2) due to 21 hydroxylase deficiency (21 OHD) and who are monitored for 52 weeks post-treatment. All participants who receive the BBP-631 treatment will be followed for an additional 4 years for safety and efficacy. In total, all participants will be followed for at least 5 years after the date of treatment with BBP-631. FIG. 2 shows the timeline for the study.

BBP-631 will be administered undiluted as a one-time, single IV infusion via volumetric infusion pump. The BBP-631 active substance is a non-replicating, recombinant AAV serotype 5 (AAV5) vector containing an expression cassette for the human CYP21A2 transgene. The AAV-hCYP21-Opt vector design is depicted in FIG. 4. The sequence of the transgene that is packaged into rAAV5 particles is a single stranded DNA comprised of the following elements listed from 5′ to 3″: AAV 5′ inverted terminal repeat (ITR), the AAV packaging signal (Y), cytomegalovirus enhancer, chicken ß-actin promoter including the canonical Kozak sequence with splice donor and rabbit β-globin intron with splice acceptor, the codon optimized human 21-hydroxylase (21-OH) cDNA, rabbit ß-globin poly A signal, and the AAV 3′ inverted terminal repeat ITR. This optimized promoter design, designated CAG, is intended to maximize expression of the transgene in host tissue.

BBP-631 drug product (DP) is a single-dose, preservative-free, sterile, opalescent, colorless solution, IV injection, of non-replicating, recombinant AAV5 vector at a target concentration of 5.0×10¹³ vg/mL (3.0×10¹³ to 7.0×10¹³ vg/mL). The BBP-631 DP solution contains 10 mM Sodium Phosphate, 180 mM Sodium Chloride, 0.005% w/v poloxamer 188 and Water for Injection. BBP-631 DP is filled into 5 mL Crystal ZenithR (CZ, cyclic olefin polymer) vials with a nominal fill volume of 2.5 mL and stored at ≤−60° C.

### Screening Period

For all participants, the Screening Period will begin at the time of the first Screening assessment. The Screening Period will be up to 42 days before treatment with BBP-631 (Day 0). Eligibility will be determined by assessment of safety and Screening laboratory samples and threshold levels of 17-OHP, androstenedione (A4), adrenocorticotropic hormone (ACTH), and cortisol. To measure 17-OHP variability, at least 3 assessments of 17-OHP will be conducted and will be separated by at least 5 days. At the times of 17-OHP assessments, samples also will be collected to assess liver function. During the Screening Period, participants will continue to take their normally scheduled oral hydrocortisone and, if applicable, mineralocorticoid treatment for CAH. Unless otherwise specified, screening samples are collected in the morning before the participants take their first normally scheduled oral hydrocortisone and, if applicable, mineralocorticoid treatment. In addition to levels of 17-OHP and androstenedione (A4), other hormone levels will be assessed including cortisol, hormones associated with the hypothalamic-pituitary-adrenal (HPA) axis, hypothalamic-pituitary-gonadal (HPG) axis, other physiologic or regulatory functions, renin and aldosterone, and steroid hormone panel. As appropriate, samples will be analyzed by immunoassay, liquid chromatography-tandem mass spectrometry (LC-MS/MS), or by both methods. Measurement of all hormone levels will be performed at a central laboratory.

### Baseline, Treatment, and Follow-Up Periods

The total duration of the Baseline, Treatment, and Follow-Up Periods is planned to be approximately 52 weeks.

### Baseline (Before Treatment With BBP-631): Inpatient (Day −4 to Day 0)

Baseline levels of other endogenous hormones, quality of life (QOL), skeletal and body composition assessments and biomarkers of bone turnover, hirsutism, ovulation, and testicular function and testicular adrenal rest tumor (TART) volume will be assessed. Unless otherwise specified, assessments will be scheduled to be conducted as close as possible to Day 0 (Dosing) to establish baseline measures. As appropriate, samples will be analyzed by immunoassay, LC-MS/MS, or by both methods. Measurement of all hormone levels will be performed at a central laboratory.

On Day −3, participants will have serial blood samples taken for assessment of cortisol clearance, ACTH, 17-OHP, and androstenedione (A4) levels. Oral hydrocortisone and, if applicable, mineralocorticoid treatment will be withheld after the regularly scheduled morning dose on Day −3.

On the morning of Day −2 and before taking their normally scheduled doses of hydrocortisone and, if applicable, mineralocorticoids, the participants will have samples taken for a hormonal profile including, but not limited to, the HPA axis and renin-aldosterone axis. These samples will serve as the baseline assessment for most hormones including, but not limited to, 17-OHP, androstenedione (A4), and endogenous ACTH. Thereafter, participants will complete an ACTH-stimulated test of endogenous morning cortisol and 17-OHP levels and, then, will take only their normally scheduled morning doses of hydrocortisone and, if applicable, mineralocorticoids. In the evening of Day −2, the diurnal hormonal profile of the participants will be established by obtaining samples every 2 hours over a 24-hour period to measure levels of HPA axis hormones including, but not limited to, cortisol, 17-OHP, androstenedione (A4), and ACTH.

On the morning of Day −2, participants will begin a prophylactic immunosuppressive regimen with tacrolimus to prevent or dampen potential immune responses after BBP-631 administration. This tacrolimus regimen will be taken orally at a starting dose of 0.025 mg/kg twice daily and will have a target trough level of 5 to 8 ng/mL. Trough levels will be measured contemporaneously with selected blood chemistry analytes.

In the evening of Day −1, after completion of the 24-hour diurnal hormonal profile sample collection period, participants will resume their normally scheduled doses of hydrocortisone and, if applicable, mineralocorticoids.

Baseline: Treatment with BBP-631 (Day 0)

On Day 0, before BBP-631 and tacrolimus dosing, tacrolimus trough levels will be measured and the participants also will receive prophylactic antihistamines, such as, diphenhydramine (IV), hydroxyzine (IM), and chlorpheniramine (IV), to prevent infusion reactions. BBP-631 will be administered as an intravenous (IV) infusion and started 2 to 6 hours after the participants regularly scheduled morning hydrocortisone dose. The participants will receive only a single administration of BBP-631.

### Follow-Up

Safety, including laboratories, immunogenicity, and vector shedding, will be evaluated continuously. Safety and efficacy data will be evaluated for decisions about dose escalation and expansion. Tacrolimus trough levels will be measured according to a schedule that maintains prophylactic immunosuppression and, if medically necessary, as part of a reactive immunosuppression plan that includes liver function assessments.

The efficacy and durability of treatment with BBP-631 will be assessed through samples obtained in both inpatient and outpatient (at-home) settings. Samples will be collected for assessment of endogenous hormones associated with the HPA axis, HPG axis, other physiologic or regulatory functions, renin and aldosterone, and steroid hormone panel. Unless otherwise specified, samples will be collected in the morning before the participants take their normally scheduled morning doses of hydrocortisone and, if applicable, mineralocorticoids. Samples also will be collected before and after a morning exogenous ACTH stimulation. Participants will have serial blood samples taken for assessment of cortisol clearance, ACTH, 17-OHP, and androstenedione (A4) levels. Additional markers including 11-oxo/11-keto sterols such as 11-ketotestosterone may also be assessed. As appropriate, samples will be analyzed by immunoassay, LC-MS/MS, or by both methods.

QOL assessments will be measured through the Short Form (36) Health Survey (SF-36), World Health Organization Quality of Life 100 Abbreviated (WHOQOL-BREF), and Addison's Disease-Specific Quality of Life Questionnaire (AddiQOL). Skeletal and body composition will be assessed via DEXA and biomarkers of bone turnover. Hirsutism in females will be assessed via the modified Ferriman-Gallwey scale and/or self-reported depilation frequency at various body regions. In females, the ovulatory cycle will be monitored. In males, testosterone (sex hormone binding globulin-adjusted) levels will be measured, presence of TARTs will be monitored by ultrasound, and testicular function will be assessed via sperm count, motility, and morphology.

### Hydrocortisone and Mineralocorticoid Treatment Tapering

Tapering of hydrocortisone treatment may be initiated at any time after treatment with BBP-631 as supported by available clinical and laboratory data, in addition to Investigator discretion.

The following tapering sequence will be used as general guidance for hydrocortisone dosing adjustments in response to participants' HPA-axis hormone levels: (1) Reduce hydrocortisone dose by up to 25% (to a minimum total daily dose of 15 mg) if: 17-OHP levels ≤1200 ng/dL, or Androstenedione (A4) levels≤normal. (2) Discontinue hydrocortisone treatment (or consider reduction below 15 mg total daily dose) if ACTH-stimulated cortisol >14 μg/dL via immunoassay. Otherwise, hydrocortisone treatment will be maintained at a minimum dose of 15 mg/day and no further reduction will take place unless ACTH-stimulated cortisol is >14 μg/dL via immunoassay.

To ensure that the participant is tolerating any hydrocortisone taper (or discontinuation): Laboratory measurements of morning 17-OHP, androstenedione (A4), cortisol, and ACTH and, optionally, 11-ketotestosterone will be obtained within 2 weeks of initiating the taper. Follow-up contact from the site to the participant to inquire about clinical status and tolerance to the taper will be added weekly for at least 2 weeks after initiating the taper.

Signs and symptoms of adrenal insufficiency will be closely monitored. If signs and symptoms become clinically evident, based on trends in clinical laboratory results and/or clinical status of the participant, it will be determines whether to increase or reinstate hydrocortisone dosing.

For participants taking mineralocorticoids, tapering is permitted only after Week 6 assessments at the discretion of the Investigator. The following tapering sequence will be used as general guidance for mineralocorticoid treatment dosing adjustments at the Investigator's discretion in response to participants' hormone levels and clinical presentation: (1) Reduce mineralocorticoid treatment by 50% if Plasma-renin activity <normal: (2) Reduce mineralocorticoid treatment in consultation if signs or symptoms of mineralocorticoid excess (eg, significant increase in blood pressure); and Consider discontinuation of mineralocorticoid treatment only if Peak plasma aldosterone concentration ≥9 ng/dL during ACTH-stimulation.

Upon tapering of mineralocorticoid dosing, signs and symptoms of mineralocorticoid deficiency will be closely monitored. During the study, mineralocorticoid treatment can be increased or reinitiated if signs and symptoms of adrenal insufficiency become clinically evident.

### Long-Term Follow-Up Period

Immediately upon completing this study, after providing consent, participants will begin a separate long-term follow-up study and be followed at appropriate intervals for an additional 4 years for continued evaluation of safety and efficacy of BBP-631 treatment. In total, all participants will be followed for at least 5 years after the date of treatment with BBP-631.

### Dose Escalation and Enrollment Expansion Scheme

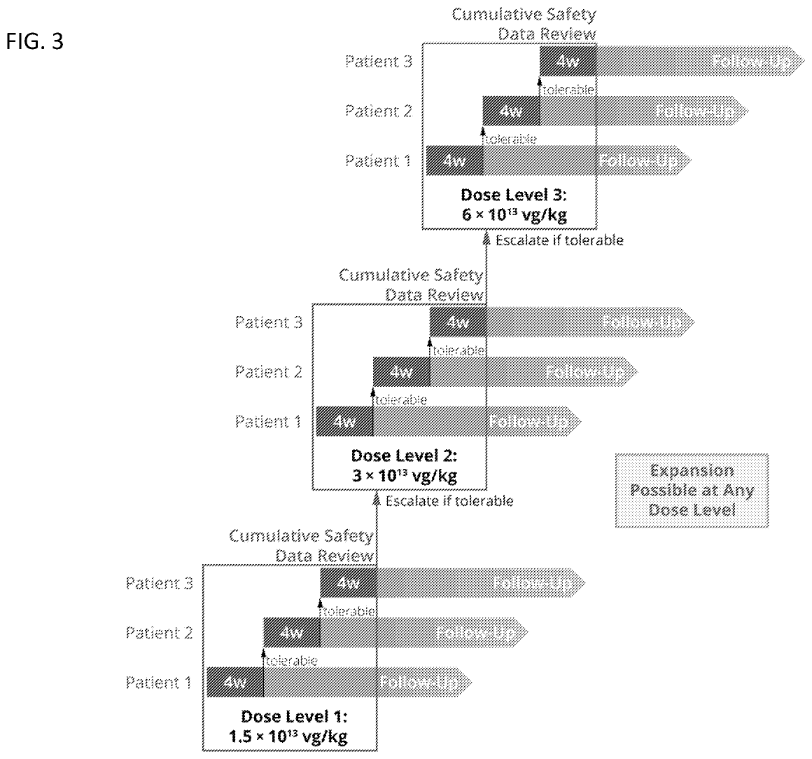

All dose levels are planned for investigation under an escalation and expansion scheme that will be determined by safety and tolerability of BBP-631. Participants will not be treated at a dose higher than Dose Level 3. See FIG. 3.

During the Dose Escalation Phase, at least 3 (and up to 6) participants from Group 1 (that is, classical CAH and on hydrocortisone doses≤30 mg/day) will be enrolled at each dose level to receive BBP-631 for a total of at least 9 (and up to 18) participants from Group 1. Dose escalation will continue based on tolerability of BBP-631. Dose expansion will be determined by tolerability and available data from Group 1 participants related to the effect of BBP-631 on HPA-axis hormones.

The sentinel participant at each BBP-631 dose level will be a participant with a non-null CYP21A2 mutation on at least 1 allele, thus reducing the potential for confounding signals that might be detected from cross-reactivity in immunological material. Non-sentinel participants at each dose level will be enrolled without any genotype exclusion. After the first participant receives BBP-631 at the starting dose (Dose Level 1), treatment of the second participant will occur only after a review of the first participant's safety data through at least the Day 28 post-dose visit. As the study proceeds, treatment of subsequent participants will be permitted after review of cumulative safety data for all participants through at least the Day 28 post-dose visit.

Reviews of at least 4 weeks of all participants' cumulative post-dosing safety data are required before escalation and before dosing the second participant in a dose cohort. The same algorithm will be followed for further dose escalation and will proceed only to Dose Level 3.

### Safety Monitoring

### Immunogenicity Monitoring and Reactive Immunomodulation Plan

Each participant will be monitored post-treatment for development of antibodies and T-cell reactivity to capsid and transgene product once per week from dosing with BBP-631 to completion of the safety assessments at Week 5, then every 3 weeks until the end of the study.

The prophylactic tacrolimus regimen will be maintained for 4 weeks after which it will be tapered over a period of approximately 2 weeks. If transaminase levels increase to ≥2× upper limit of the normal range (ULN) or to clinically relevant levels in the opinion of the Investigator at any time during the study, then tacrolimus dosing either will be restarted (targeting trough levels) or increased. In addition, high-dose prednisone (or, at the Investigator's discretion, hydrocortisone) will be started and, when transaminase levels normalize, followed by a rapid taper over a 3-week period to baseline glucocorticoid dose levels. Concurrently, liver function tests (LFTs) will be monitored weekly or more frequently until the LFT levels return to ≤ ULN. During this monitoring period, tacrolimus and prednisone dosing may be adjusted in response to observed LFT levels. If the participant has discontinued glucocorticoids for 4 weeks with LFTs normalized, then tacrolimus tapering may resume. If necessary, a treatment plan involving increases in tacrolimus target trough levels or administration of other immunomodulating agents may be started.

### Other Considerations

Any hepatotoxic agents (such as, ketamine, halogenated anesthetics) are prohibited for at least 12 weeks after receiving BBP-631. The participant will be advised to avoid taking acetaminophen (and other similar medications) during the same period of time. Participants should avoid substances that inhibit or induce tacrolimus metabolism via the CYP3A or CYP3A4 pathway, including grapefruit juice.

A baseline (5-day assessment period, such as a 5-day inpatient stay) with a detailed assessment of diurnal hormonal profile (including 17-OHP and A4), cortisol clearance, ACTH-stimulation testing, renin and aldosterone, and other exploratory hormones will be performed.

The key eligibility criteria for patient population include the following. The patient has CAH diagnosis: Group 1: Diagnosis of classical CAH (simple virilizing or salt-wasting) since childhood based on clinical and genetic evidence, including well-documented CAH historical data (ie, medical history, longitudinal laboratory values, and medication history), or Group 2: Diagnosis of classical, salt-wasting CAH since childhood based on clinical and genetic evidence, including well-documented CAH historical data (ie, medical history, longitudinal laboratory values, and medication history). The patient meets threshold 17-OHP levels at the initial Screening visit: Group 1: 17-OHP levels ≥10×ULN (upper limit of normal) but <40×ULN, or Group 2: 17-OHP levels ≥5×ULN but <40×ULN. The patient has androstenedione (A4) value above ULN. The patient is on a stable regimen of oral hydrocortisone as the only glucocorticoid maintenance therapy at the following total daily doses: Group 1: ≤30 mg/day hydrocortisone for treatment of CAH for 3 months before Screening, or Group 2: >30 mg/day hydrocortisone for treatment of CAH for 3 months before Screening. If the patient is already taking mineralocorticoid agonist therapy, then the patient has a documented history of requiring mineralocorticoid agonist therapy (eg, fludrocortisone) and on stable dose of this mineralocorticoid agonist for 3 months before Screening.

### Dosage

Three dose levels of BBP-631 will be used in the study: (i) Dose Level 1: 1.5×10¹³ vg/kg body weight: (ii) Dose Level 2: 3.0×10¹³ vg/kg body weight, and (iii) Dose Level 3: 6.0×10¹³ vg/kg body weight. Each treatment-eligible participant will receive a single dose of BBP-631 administered by IV infusion on Day 0 of the study. The duration of the infusion will depend on the treatment volume that is calculated according to the participant's body weight.

### Safety and Efficacy Analyses

The optimum dose will be selected based on the following: (i) Adverse Events (AEs), clinical laboratory measures (chemistry, hematology, urinalysis): (ii) levels of endogenous cortisol (pre- and post-ACTH stimulation, measured via both mass spectrometry and immunoassay), 17-OHP, A4, and other hormones associated with the Hypothalamic-Pituitary-Adrenal (HPA) and Hypothalamic-Pituitary-Gonadal (HPG) axes: (iii) levels of renin and aldosterone: (iv) changes in hydrocortisone (HC) and mineralocorticoid (MC) medication use: and (v) quality of life assessments measuring physical and physiological impacts of the hormonal imbalance.

Clinically significant changes in physical examination findings will be reported as AEs. To evaluate the safety and tolerability of BBP-631 in adult participants with classical CAH, clinical laboratory assessments (clinical chemistry, coagulation, hematology, and urinalysis) will be collected. Laboratory and vital signs, 12-lead ECGs, and physical examination findings will be collected. To select the optimum dose or dose range of BBP-631 for future studies, safety and tolerability assessments, and hormonal and other efficacy assessments will be done.

The following safety and efficacy evaluations will be evaluated as listed below:

TABLE 2

| | Experimental Determination of the |
|---|---|
| Safety and/or Efficacy evaluation | safety/efficacy variable |
| To evaluate the safety and tolerability | AEs, SAEs, deaths, and discontinuations due to |
| of BBP-631 in adult participants with | AEs |
| classical CAH | Clinical laboratory tests (hematology, coagulation, |
| | chemistry, and urinalysis) |
| | Vital signs, 12-lead ECGs, and physical |
| | examination findings |
| To select the optimum dose or dose | Safety and tolerability assessments |
| range of BBP-631 for future studies | Hormonal and other efficacy assessments |
| To evaluate the effect of BBP-631 on | Change from Pretreatment to Week 52 in 17-OHP |
| 17-OHP levels in adult participants | levels |
| with classical CAH (Group 1) | |
| To evaluate the effect of BBP-631 on | Change from Pretreatment to Week 52 in |
| endogenous cortisol production in | endogenous cortisol levels (exogenous steroids |
| adult participants with classical CAH | held) as measured both pre- and post-stimulation |
| | with exogenous ACTH |
| To evaluate the effect of BBP-631 on | Change from Pretreatment to Week 52 in |
| androstenedione (A4) production in | androstenedione (A4) |
| adult participants with classical CAH | |
| To evaluate the effect of BBP-631 on | Change from Pretreatment to Week 52 in |
| the renin-aldosterone pathway in | aldosterone and renin (mineralocorticoid held) |
| adult participants with classical CAH | measured concurrently with endogenous cortisol |
| | (exogenous steroids held) as measured both pre- |
| | and post-stimulation with exogenous ACTH |
| To evaluate immunogenicity of BBP- | Biomarkers of immunogenicity |
| 631 in in adult participants with | |
| classical CAH | |
| To evaluate the effect of BBP-631 on | Change from Pretreatment to Week 52 in DHEA, |
| other adrenal androgens in adult | DHEA-S (men and women), and testosterone |
| participants with classical CAH | (women only) |
| To evaluate the effect of BBP-631 on | Change from Pretreatment to Week 52 in ACTH |
| ACTH in adult participants with | |
| classical CAH | |
| To evaluate the effect of BBP-631 on | Change from Pretreatment to Week 52 in full panel |
| other adrenal hormones in adult | of D4 and D5 steroids |
| participants with classical CAH | |
| To evaluate the effect of BBP-631 on | Change from Pretreatment to Week 52 in cortisol |
| cortisol clearance | clearance |
| To evaluate changes in oral | Proportion of participants reducing or eliminating |
| glucocorticoid use after treatment | oral glucocorticoid treatment at Week 52 following |
| with BBP-631 | BBP-631 infusion |
| To evaluate changes in oral | Proportion of participants reducing or eliminating |
| mineralocorticoid use after treatment | oral mineralocorticoid treatment at Week 52 |
| with BBP-631 | following BBP-631 infusion |
| To evaluate the effect of BBP-631 on | Change from Pretreatment in menstrual status |
| ovulatory cycle (women of | |
| childbearing potential who are not | |
| using exogenous medications that | |
| impact the menstrual cycle [eg, oral | |
| contraceptive medication]) | |
| To evaluate changes in perceived | Change from Pretreatment in SF-36 scores |
| QOL after administration of BBP-631 | Change from Pretreatment in WHOQOL-BREF |
| | scores |
| | Change from Pretreatment in AddiQOL |
| To evaluate the change in diurnal | Change from Pretreatment of cortisol levels as |
| pattern of endogenous glucocorticoid | measured over a 24-hour period, 1 day after holding |
| production | exogenous steroids |
| To evaluate the effects of BBP-631 | Change from Pretreatment to Week 52 in LH, FSH, |
| on HPG axis | AMH, and inhibin in all participants; estradiol (E2) |
| | and progesterone in female participants; and |
| | testosterone (sex hormone binding globulin- |
| | adjusted) in male participants |
| To evaluate the effect of BBP-631 on | Change from Pretreatment to Week 52 in fasting |
| insulin and glucose homeostasis | glucose, fasting insulin, and associated measures of |
| | insulin sensitivity (HOMA-IR) |
| To evaluate the effect of BBP-631 on | Change from Pretreatment to Week 52 in fat and |
| body composition | lean mass as measured by DEXA |
| To evaluate the effect of BBP-631 on | Change from Pretreatment to Week 52 in bone |
| bone density | mineral density at the total hip, femoral neck and |
| | lumbar spine as measured by DEXA |
| To evaluate the effect of BBP-631 on | Change from Pretreatment to Week 52 in serum |
| bone turnover markers | CTX, serum NTX, and serum PINP |
| To evaluate the effect of BBP-631 on | Change from Pretreatment in modified Ferriman- |
| hirsutism (females only) | Gallwey scores (hirsutism) and/or self-reported |
| | depilation frequency at various body regions |
| | (females only) |
| To evaluate the effect of BBP-631 on | Changes from Pretreatment in testicular volume |
| TART volume (males only) | including TART size by ultrasound, as appropriate |
| | (males only) |
| To evaluate the effect of BBP-631 on | Changes from Pretreatment in sperm count, |
| spermatogenesis (males only) | motility, and morphology (males only) |

Abbreviations: ACTH: adrenocorticotropic hormone; AddiQOL: Addison's Disease-Specific Quality of Life Questionnaire; AE: adverse event; AMH: anti-Mullerian hormone; CAH: congenital adrenal hyperplasia; CTX: carboxy-terminal collagen crosslinks (C-terminal telopeptide); DEXA: dual-energy x-ray absorptiometry; DHEA: dehydroepiandrosterone; DHEA-S: dehydroepiandrosterone-sulfate; ECG: electrocardiogram; FSH: follicle stimulating hormone; HOMA-IR: homeostatic model assessment of insulin resistance; HPA: hypothalamic-pituitary-adrenal; HPG: hypothalamic-pituitary-gonadal; LH: luteinizing hormone NTX: amino-terminal collagen crosslinks (serum N-terminal telopeptide); OHP: hydroxyprogesterone; PINP: serum N terminal propeptide of type I procollagen; QOL: quality of life; SAE: serious adverse event; SF-36: Short Form (36) Health Survey; TART: testicular adrenal rest tumor; WHOQOL-BREF: World Health Organization Quality of Life 100 Abbreviated
Note:
Changes in assessment variables from pretreatment values may be determined by using measurements obtained during Screening or at the study Baseline timepoint.

### Numbered Embodiments

Embodiment 1. A method, comprising:
administering to a subject a therapeutically effective amount of a recombinant adeno-associated virus (rAAV) vector, wherein the rAAV vector comprises:
   (i) a nucleic acid molecule comprising at least one AAV inverted terminal repeat (ITR) and
   (ii) a non-AAV nucleotide sequence encoding a 21-hydroxylase (21OH) protein, wherein the non-AAV nucleotide sequence is operably linked to a promoter; and
wherein the therapeutically effective amount is in the range of about 10¹² vg/kg to about 10¹⁴ vg/kg.

Embodiment 2. The method of embodiment 1, wherein the subject is in need of expression of 21OH.
Embodiment 3. The method of embodiment 1 or 2, wherein the subject has a 21OH deficiency (21OHD).

Embodiment 4. The method of embodiment 3, wherein the subject has the Prader stage IV or V form of 21OHD.

Embodiment 5. The method of embodiment 3 or 4, wherein the subject has congenital adrenal hyperplasia (CAH).

Embodiment 6. The method of embodiment 5, wherein the CAH is classical CAH.

Embodiment 7. The method of any one of embodiments 1-6, wherein administration of the rAAV vector results in expression of 21OH in the subject.

Embodiment 8. The method of embodiment 7, wherein the 21OH is expressed in the subject's adrenal cortex, adrenal medulla, adrenal stem cells, adrenal progenitor cells, liver, or ovary.

Embodiment 9. The method of any one of embodiments 1-8, wherein the subject is in need of cortisol and/or aldosterone.

Embodiment 10. The method of any one of embodiments 1-9, wherein the subject has a cortisol deficiency and/or an aldosterone deficiency.

Embodiment 11. The method of any one of embodiments 1-10, wherein the subject has an excess of progesterone, 17-OHP, renin, and/or androstenedione (A4).

Embodiment 12. The method of embodiment 11, wherein the subject has an excess of progesterone, 17-OHP, renin, and/or androstenedione (A4) in the blood and/or urine.

Embodiment 13. A method of expressing 21-hydroxlase (21OH) in a subject in need thereof, comprising:
administering to the subject a therapeutically effective amount of a recombinant adeno-associated virus (rAAV) vector, wherein the rAAV vector comprises:
   (i) a nucleic acid molecule comprising at least one AAV inverted terminal repeat (ITR) and
   (ii) a non-AAV nucleotide sequence encoding a 21-hydroxylase (21OH) protein, wherein the non-AAV nucleotide sequence is operably linked to a promoter; and
wherein the therapeutically effective amount is in the range of about 10¹² vg/kg to about 10¹⁴ vg/kg, thereby expressing 21OH in the subject.

Embodiment 14. The method of embodiment 13, wherein the 21OH is expressed in the subject's adrenal cortex, adrenal medulla, adrenal stem cells, adrenal progenitor cells, liver, or ovary.

Embodiment 15. A method of treating a subject having 21-hydroxylase deficiency (21OHD), comprising: administering to the subject a therapeutically effective amount of a recombinant adeno-associated virus (rAAV) vector, wherein the rAAV vector comprises:

(i) a nucleic acid molecule comprising at least one AAV inverted terminal repeat (ITR) and

(ii) a non-AAV nucleotide sequence encoding a 21-hydroxylase (21OH) protein, wherein the non-AAV nucleotide sequence is operably linked to a promoter; and wherein the therapeutically effective amount is in the range of about 10¹² vg/kg to about 10¹⁴ vg/kg, thereby treating 21OHD in the subject,

Embodiment 16. A method of increasing the level of cortisol and/or aldosterone in a subject in need thereof, comprising: administering to the subject a therapeutically effective amount of a recombinant adeno-associated virus (rAAV) vector, wherein the rAAV vector comprises:
(i) a nucleic acid molecule comprising at least one AAV inverted terminal repeat (ITR) and
(ii) a non-AAV nucleotide sequence encoding a 21-hydroxylase (21OH) protein, wherein the non-AAV nucleotide sequence is operably linked to a promoter; and wherein the therapeutically effective amount is in the range of about 10¹² vg/kg to about 10¹⁴ vg/kg, thereby increasing the level of cortisol and/or aldosterone in the subject.

Embodiment 17. A method of decreasing the level of progesterone, 17-OHP, renin and/or androstenedione (A4) in a subject in need thereof, comprising: administering to the subject a therapeutically effective amount of a recombinant adeno-associated virus (rAAV) vector, wherein the rAAV vector comprises:
(i) a nucleic acid molecule comprising at least one AAV inverted terminal repeat (ITR) and
(ii) a non-AAV nucleotide sequence encoding a 21-hydroxylase (21OH) protein, wherein the non-AAV nucleotide sequence is operably linked to a promoter; and wherein the therapeutically effective amount is in the range of about 10¹² vg/kg to about 10¹⁴ vg/kg, thereby decreasing the level of progesterone, 17-OHP, renin and/or androstenedione (A4) in the subject.

Embodiment 18. The method of any one of embodiments 13-17, wherein the subject has the Prader stage IV or V form of 21-hydroxylase deficiency (21OHD).

Embodiment 19. The method of any one of embodiments 13-17, wherein the subject has congenital adrenal hyperplasia (CAH).

Embodiment 20. The method of embodiment 19, wherein the CAH is classical CAH.

Embodiment 21. The method of any one of embodiments 1-20, wherein the therapeutically effective amount is in the range of about 10¹³ vg/kg to about 10¹⁴ vg/kg.

Embodiment 22. The method of any one of embodiments 1-20, wherein the therapeutically effective amount is about 1.5×10¹³ vg/kg.

Embodiment 23. The method of any one of embodiments 1-20, wherein the therapeutically effective amount is about 3×10¹³ vg/kg.

Embodiment 24. The method of any one of embodiments 1-20, wherein the therapeutically effective amount is about 6×10¹³ vg/kg.

Embodiment 25. The method of any one of embodiments 1-24, further comprising selecting a subject with 21-hydroxylase deficiency (21OHD) before the administering step.

Embodiment 26. The method of any one of embodiments 1-25, wherein the rAAV vector is administered to the subject intravenously, by direct injection into the adrenal gland via open surgery or laparoscopy, or by injection into an adrenal artery or an adrenal vein via catheterization.

Embodiment 27. The method of embodiment 26, wherein the direct injection into the adrenal gland is direct injection into the adrenal cortex.

Embodiment 28. The method of any one of embodiments 1-25, wherein the rAAV vector is administered by intravenous (IV) infusion.

Embodiment 29. The method of any one of embodiments 1-28, wherein the 21OH protein is human 21OH protein.

Embodiment 30. The method of any one of embodiments 1-29, wherein the non-AAV nucleotide sequence encoding a 21OH protein comprises or consists of the human 21OH (CYP21A2) cDNA.

Embodiment 31. The method of any one of embodiments 1-30, wherein the non-AAV nucleotide sequence encoding a 21OH protein comprises or consists of a codon-optimized nucleotide sequence.

Embodiment 32. The method of any one of embodiments 1-31, wherein the non-AAV nucleotide sequence encoding a 21OH protein comprises or consists of SEQ ID NO: 50.

Embodiment 33. The method of any one of embodiments 1-32, wherein the non-AAV nucleotide sequence encoding a 21OH protein encodes the amino acid sequence of SEQ ID NO: 1 or an amino acid sequence at least 95%, at least 96%, at least 97%, at least 98% or at least 99% identical to SEQ ID NO:1.

Embodiment 34. The method of any one of embodiments 1-33, wherein the promoter directs expression of the 21OH protein in a host cell.

Embodiment 35. The method of embodiment 34, wherein the host cell is an adrenal gland cell or an adrenal cortex cell.

Embodiment 36. The method of any one of embodiments 1-35, wherein the promoter is a cytomegalovirus/β-actin hybrid promoter, PGK promoter, or a promoter specific for expression in an adrenal cortex cell.

Embodiment 37. The method of embodiment 36, wherein the cytomegalovirus/β-actin hybrid promoter is a CAG, CB6, or CBA promoter.

Embodiment 38. The method of any one of embodiments 1-37, wherein the promoter comprises or consists of the nucleotide sequence of SEQ ID NO: 2, SEQ ID NO: 3, SEQ ID NO:48, or SEQ ID NO:49.

Embodiment 39. The method of any one of embodiments 1-38, wherein the ITR is an AAV1, AAV2, AAV3, AAV4, AAV5, AAV6, AAV7, AAV8, AAV9, AAV10, AAV11, AAV12, rh10, or rh74 serotype ITR.

Embodiment 40. The method of any one of embodiments 1-39, wherein the rAAV is an AAV1, AAV2, AAV3, AAV4, AAV5, AAV6, AAV7, AAV8, AAV9, AAV10, AAV11, AAV12, rh10, or rh74 serotype.

Embodiment 41. The method of any one of embodiments 1-39, wherein the rAAV is an AAV5 serotype.

Embodiment 42. The method of any one of embodiments 1-41, wherein the nucleic acid molecule further comprises a Kozak sequence.

Embodiment 43. The method of any one of embodiments 1-42, wherein the nucleic acid molecule further comprises an miR-122 binding site.

Embodiment 44. A method, comprising: administering to a subject about 10¹² vg/kg to about 10¹⁴ vg/kg of a recombinant adeno-associated virus 5 (rAAV5) vector, wherein the rAAV5 vector comprises: a nucleic acid molecule comprising at least one AAV inverted terminal repeat (ITR), and a non-AAV nucleotide sequence encoding the amino acid sequence of SEQ ID NO: 1, wherein the non-AAV nucleotide sequence is operably linked to a promoter that directs expression of the 21OH protein in an adrenal gland cell or an adrenal cortex cell.

Embodiment 45. The method of embodiment 44, wherein about 10¹³ vg/kg to about 10¹⁴ vg/kg of the rAAV5 vector is administered to the subject.

Embodiment 46. The method of embodiment 45, wherein about 1.5 ×10¹³ vg/kg of the rAAV5 vector is administered to the subject.

Embodiment 47. The method of embodiment 45, wherein about 3 ×10¹³ vg/kg of the rAAV5 vector is administered to the subject.

Embodiment 48. The method of embodiment 45, wherein about 6 ×10¹³ vg/kg of the rAAV5 vector is administered to the subject.

Embodiment 49. The method of any one of embodiments 45-48, wherein the rAAV5 vector is administered to the subject intravenously.

Embodiment 50. The method of any one of embodiments 44-49, wherein the subject has 21-hydroxylase deficiency (21OHD).

Embodiment 51. A method of treating a subject having 21-hydroxylase deficiency (21OHD), comprising: intravenously administering to the subject about 1.5 ×10¹³ vg/kg of a recombinant adeno-associated virus 5 (rAAV5) vector, wherein the rAAV5 vector comprises: a nucleic acid molecule comprising at least one AAV inverted terminal repeat (ITR), and a non-AAV nucleotide sequence encoding the amino acid sequence of SEQ ID NO: 1, wherein the non-AAV nucleotide sequence is operably linked to a promoter that directs expression of the 21OH protein in an adrenal gland cell or an adrenal cortex cell, thereby treating 21OHD in the subject.

Embodiment 52. A method of treating a subject having 21-hydroxylase deficiency (21OHD), comprising: intravenously administering to the subject about 3 ×10¹³ vg/kg of a recombinant adeno-associated virus 5 (rAAV5) vector, wherein the rAAV5 vector comprises: a nucleic acid molecule comprising at least one AAV inverted terminal repeat (ITR), and a non-AAV nucleotide sequence encoding the amino acid sequence of SEQ ID NO: 1, wherein the non-AAV nucleotide sequence is operably linked to a promoter that directs expression of the 21OH protein in an adrenal gland cell or an adrenal cortex cell, thereby treating 21OHD in the subject.

Embodiment 53. A method of treating a subject having 21-hydroxylase deficiency (21OHD), comprising: intravenously administering to the subject about 6 ×10¹³ vg/kg of a recombinant adeno-associated virus 5 (rAAV5) vector, wherein the rAAV5 vector comprises:
(i) a nucleic acid molecule comprising at least one AAV inverted terminal repeat (ITR), and
(ii) a non-AAV nucleotide sequence encoding the amino acid sequence of SEQ ID NO: 1, wherein the non-AAV nucleotide sequence is operably linked to a promoter that directs expression of the 21OH protein in an adrenal gland cell or an adrenal cortex cell, thereby treating 21OHD in the subject.

Embodiment 54. The method of any one of embodiments 44-53, wherein the promoter is a cytomegalovirus/β-actin hybrid promoter, PGK promoter, or a promoter specific for expression in an adrenal cortex cell.

Embodiment 55. The method of embodiment 54, wherein the cytomegalovirus/β-actin hybrid promoter is a CAG, CB6, or CBA promoter.

Embodiment 56. The method of any one of embodiments 44-55, wherein the promoter comprises or consists of the nucleotide sequence of SEQ ID NO: 2, SEQ ID NO: 3, SEQ ID NO:48, or SEQ ID NO:49.

Embodiment 57. The method of any one of embodiments 44-56, wherein the ITR is an AAV1, AAV2, AAV3, AAV4, AAV5, AAV6, AAV7, AAV8, AAV9, AAV10, AAV11, AAV12, rh10, or rh74 serotype ITR.

Embodiment 58. The method of any one of embodiments 44-57, wherein the nucleic acid molecule further comprises a Kozak sequence.

Embodiment 59. The method of any one of embodiments 44-58, wherein the nucleic acid molecule further comprises an miR-122 binding site.

Embodiment 60. The method of any one of embodiments 44-59, wherein the subject has the Prader stage IV or V form of 21-hydroxylase deficiency (21OHD).

Embodiment 61. The method of any one of embodiments 44-59 wherein the subject has congenital adrenal hyperplasia (CAH).

Embodiment 62. The method of embodiment 61, wherein the CAH is classical CAH.

Embodiment 63. The method of any one of embodiments 1-62, further comprising administering a therapeutically effective amount of an immunosuppressant to the subject.

Embodiment 64. A method, comprising: administering to the subject a therapeutically effective amount of a recombinant adeno-associated virus 5 (rAAV5) vector and a therapeutically effective amount of an immunosuppressant, wherein the rAAV5 comprises a nucleic acid molecule comprising: at least one AAV inverted terminal repeat (ITR) and a non-AAV nucleotide sequence encoding the amino acid sequence of SEQ ID NO: 1, wherein the non-AAV nucleotide sequence is operably linked to a promoter that directs expression of the 21OH protein in an adrenal gland cell or an adrenal cortex cell.

Embodiment 65. The method of embodiment 64, wherein the subject has 21-hydroxylase deficiency (21OHD).

Embodiment 66. A method of treating a subject having 21-hydroxylase deficiency (21OHD), comprising: intravenously administering to the subject a therapeutically effective amount of a recombinant adeno-associated virus 5 (rAAV5) vector and a therapeutically effective amount of an immunosuppressant, wherein the rAAV5 comprises a nucleic acid molecule comprising: at least one AAV inverted terminal repeat (ITR) and a non-AAV nucleotide sequence encoding the amino acid sequence of SEQ ID NO: 1, wherein the non-AAV nucleotide sequence is operably linked to a promoter that directs expression of the 21OH protein in an adrenal gland cell or an adrenal cortex cell, thereby treating 21OHD in the subject.

Embodiment 67. The method of any one of embodiments 63-66, wherein the immunosuppressant is administered before, concurrently with, and/or after the administration of the rAAV vector.

Embodiment 68. The method of embodiment 67, wherein the immunosuppressant is administered before the administration of the rAAV vector.

Embodiment 69. The method of embodiment 68, wherein the immunosuppressant is administered at least 12 hours before the administration of the rAAV vector.

Embodiment 70. The method of embodiment 69, wherein the immunosuppressant is administered about 2 days before the administration of the rAAV vector.

Embodiment 71. The method of any one of embodiments 63-70, wherein the immunosuppressant is a non-glucocorticoid immunosuppressant.

Embodiment 72. The method of any one of embodiments 63-70, wherein the immunosuppressant is an inhibitor of calcineurin.

Embodiment 73. The method of any one of embodiments 63-72, wherein the immunosuppressant is ciclosporin, tacrolimus, sirolimus, everolimus, zotarolimus, or any combination thereof.

Embodiment 74. The method of embodiment 73, wherein the immunosuppressant is tacrolimus.

Embodiment 75. The method of any one of embodiments 63-74, wherein the immunosuppressant is administered orally.

Embodiment 76. The method of any one of embodiments 63-75, wherein the therapeutically effective amount of the immunosuppressant is in the range of about 0.01 mg/kg to about 0.05 mg/kg.

Embodiment 77. The method of embodiment 76, wherein the therapeutically effective amount of the immunosuppressant is about 0.025 mg/kg.

Embodiment 78. The method of any one of embodiments 63-77, wherein the therapeutically effective amount of the immunosuppressant is administered twice daily.

Embodiment 79. The method of any one of embodiments 1-78, comprising administering a therapeutically effective amount of a steroid to the subject.

Embodiment 80. The method of embodiment 79, wherein the steroid is a mineralocorticoid.

Embodiment 81. The method of embodiment 79, wherein the steroid is a glucocorticoid.

Embodiment 82. The method of embodiment 79, wherein the steroid is hydrocortisone.

Embodiment 83. The method of any one of embodiments 79-82, wherein the steroid is administered before, concurrently with, and/or after administration of the rAAV vector.

Embodiment 84. The method of embodiment 83, wherein the therapeutically effective amount of the steroid administered to the subject before the administration of the rAAV vector is higher than the therapeutically effective amount of the steroid administered to the subject after the administration of the rAAV.

Embodiment 85. The method of any one of embodiments 1-84, wherein there is a higher level of cortisol and/or aldosterone in the subject after administration of the rAAV, as compared to before administration of the rAAV.

Embodiment 86. The method of any one of embodiments 1-85, wherein there is a higher level of cortisol and/or aldosterone in the subject after administration of the rAAV, as compared to a control subject having 21-hydroxylase deficiency (21OHD), who has not been administered the rAAV.

Embodiment 87. The method of any one of embodiments 1-86, wherein there is a lower level of progesterone, 17-OHP, renin and/or androstenedione (A4) in the subject after administration of the rAAV, as compared to before administration of the rAAV.

Embodiment 88. The method of any one of embodiments 1-87, wherein there is a lower level of progesterone, 17-OHP, renin and/or androstenedione (A4) in the subject after administration of the rAAV, as compared to a control subject having 21-hydroxylase deficiency (21OHD), who has not been administered the rAAV.

Embodiment 89. The method of any one of embodiments 1-88, wherein the rAAV vector comprises an expression cassette comprising the nucleic acid sequence of SEQ ID NO: 52.

What is claimed is:

1. A method, comprising:
administering to a subject a therapeutically effective amount of a recombinant adeno-associated virus (rAAV) vector, wherein the rAAV vector comprises:
(i) a nucleic acid molecule comprising at least one AAV inverted terminal repeat (ITR) and
(ii) a non-AAV nucleotide sequence encoding a 21-hydroxylase (21OH) protein, wherein the non-AAV nucleotide sequence is operably linked to a promoter; and
wherein the therapeutically effective amount is in the range of about 10¹² vector genomes per kilogram (vg/kg) to about 10¹⁴ vg/kg.

2. The method of claim 1, wherein the subject is in need of expression of 21OH.

3. The method of claim 1 or 2, wherein the subject has a 21OH deficiency (21OHD).

4. The method of claim 3, wherein the subject has the Prader stage IV or V form of 21OHD.

5. The method of claim 3 or 4, wherein the subject has congenital adrenal hyperplasia (CAH).

6. The method of claim 5, wherein the CAH is classical CAH.

7. The method of any one of claims 1-6, wherein administration of the rAAV vector results in expression of 21OH in the subject.

8. The method of claim 7, wherein the 21OH is expressed in the subject's adrenal cortex, adrenal medulla, adrenal stem cells, adrenal progenitor cells, liver, or ovary.

9. The method of any one of claims 1-8, wherein the subject is in need of cortisol and/or aldosterone.

10. The method of any one of claims 1-9, wherein the subject has a cortisol deficiency and/or an aldosterone deficiency.

11. The method of any one of claims 1-10, wherein the subject has an excess of progesterone, 17-OHP, renin, and/or androstenedione (A4).

12. The method of claim 11, wherein the subject has an excess of progesterone, 17-OHP, renin, and/or androstenedione (A4) in the blood and/or urine.

13. A method of expressing 21-hydroxlase (21OH) in a subject in need thereof, comprising:
administering to the subject a therapeutically effective amount of a recombinant adeno-associated virus (rAAV) vector, wherein the rAAV vector comprises:
(i) a nucleic acid molecule comprising at least one AAV inverted terminal repeat (ITR) and
(ii) a non-AAV nucleotide sequence encoding a 21-hydroxylase (21OH) protein, wherein the non-AAV nucleotide sequence is operably linked to a promoter; and
wherein the therapeutically effective amount is in the range of about 10¹² vector genomes per kilogram (vg/kg) to about 10¹⁴ vg/kg,
thereby expressing 21OH in the subject.

14. The method of claim 13, wherein the 21OH is expressed in the subject's adrenal cortex, adrenal medulla, adrenal stem cells, adrenal progenitor cells, liver, or ovary.

15. A method of treating a subject having 21-hydroxylase deficiency (21OHD), comprising:
administering to the subject a therapeutically effective amount of a recombinant adeno-associated virus (rAAV) vector, wherein the rAAV vector comprises:
(i) a nucleic acid molecule comprising at least one AAV inverted terminal repeat (ITR) and
(ii) a non-AAV nucleotide sequence encoding a 21-hydroxylase (21OH) protein, wherein the non-AAV nucleotide sequence is operably linked to a promoter; and
wherein the therapeutically effective amount is in the range of about 10¹² vector genomes per kilogram (vg/kg) to about 10¹⁴ vg/kg,
thereby treating 21OHD in the subject.

16. A method of increasing the level of cortisol and/or aldosterone in a subject in need thereof, comprising:
administering to the subject a therapeutically effective amount of a recombinant adeno-associated virus (rAAV) vector, wherein the rAAV vector comprises:
(i) a nucleic acid molecule comprising at least one AAV inverted terminal repeat (ITR) and
(ii) a non-AAV nucleotide sequence encoding a 21-hydroxylase (21OH) protein, wherein the non-AAV nucleotide sequence is operably linked to a promoter; and
wherein the therapeutically effective amount is in the range of about 10¹² vector genomes per kilogram (vg/kg) to about 10¹⁴ vg/kg,
thereby increasing the level of cortisol and/or aldosterone in the subject.

17. A method of decreasing the level of progesterone, 17-OHP, renin and/or androstenedione (A4) in a subject in need thereof, comprising:
administering to the subject a therapeutically effective amount of a recombinant adeno-associated virus (rAAV) vector, wherein the rAAV vector comprises:
(i) a nucleic acid molecule comprising at least one AAV inverted terminal repeat (ITR) and
(ii) a non-AAV nucleotide sequence encoding a 21-hydroxylase (21OH) protein, wherein the non-AAV nucleotide sequence is operably linked to a promoter; and
wherein the therapeutically effective amount is in the range of about 10¹² vector genomes per kilogram (vg/kg) to about 10¹⁴ vg/kg,
thereby decreasing the level of progesterone, 17-OHP, renin and/or androstenedione (A4) in the subject.

18. The method of any one of claims 13-17, wherein the subject has the Prader stage IV or V form of 21-hydroxylase deficiency (21OHD).

19. The method of any one of claims 13-17, wherein the subject has congenital adrenal hyperplasia (CAH).

20. The method of claim 19, wherein the CAH is classical CAH.

21. The method of any one of claims 1-20, wherein the therapeutically effective amount is in the range of about 10¹³ vg/kg to about 10¹⁴ vg/kg.

22. The method of any one of claims 1-20, wherein the therapeutically effective amount is about 1.5 ×10¹³ vg/kg.

23. The method of any one of claims 1-20, wherein the therapeutically effective amount is about 3 ×10¹³ vg/kg.

24. The method of any one of claims 1-20, wherein the therapeutically effective amount is about 6 ×10¹³ vg/kg.

25. The method of any one of claims 1-24, further comprising selecting a subject with 21-hydroxylase deficiency (21OHD) before the administering step.

26. The method of any one of claims 1-25, wherein the rAAV vector is administered to the subject intravenously, by direct injection into the adrenal gland via open surgery or laparoscopy, or by injection into an adrenal artery or an adrenal vein via catheterization.

27. The method of claim 26, wherein the direct injection into the adrenal gland is direct injection into the adrenal cortex.

28. The method of any one of claims 1-25, wherein the rAAV vector is administered by intravenous (IV) infusion.

29. The method of any one of claims 1-28, wherein the 21OH protein is human 21OH protein.

30. The method of any one of claims 1-29, wherein the non-AAV nucleotide sequence encoding a 21OH protein comprises or consists of the human 21OH (CYP21A2) cDNA.

31. The method of any one of claims 1-30, wherein the non-AAV nucleotide sequence encoding a 21OH protein comprises or consists of a codon-optimized nucleotide sequence.

32. The method of any one of claims 1-31, wherein the non-AAV nucleotide sequence encoding a 21OH protein comprises or consists of SEQ ID NO: 50.

33. The method of any one of claims 1-32, wherein the non-AAV nucleotide sequence encoding a 21OH protein encodes the amino acid sequence of SEQ ID NO: 1 or an amino acid sequence at least 95%, at least 96%, at least 97%, at least 98% or at least 99% identical to SEQ ID NO:1.

34. The method of any one of claims 1-33, wherein the promoter directs expression of the 21OH protein in a host cell.

35. The method of claim 34, wherein the host cell is an adrenal gland cell or an adrenal cortex cell.

36. The method of any one of claims 1-35, wherein the promoter is a cytomegalovirus/β-actin hybrid promoter, PGK promoter, or a promoter specific for expression in an adrenal cortex cell.

37. The method of claim 36, wherein the cytomegalovirus/β-actin hybrid promoter is a CAG, CB6, or CBA promoter.

38. The method of any one of claims 1-37, wherein the promoter comprises or consists of the nucleotide sequence of SEQ ID NO: 2, SEQ ID NO: 3, SEQ ID NO:48, or SEQ ID NO:49.

39. The method of any one of claims 1-38, wherein the ITR is an AAV1, AAV2, AAV3, AAV4, AAV5, AAV6, AAV7, AAV8, AAV9, AAV10, AAV11, AAV12, rh10, or rh74 serotype ITR.

40. The method of any one of claims 1-39, wherein the rAAV is an AAV1, AAV2, AAV3, AAV4, AAV5, AAV6, AAV7, AAV8, AAV9, AAV10, AAV11, AAV12, rh10, or rh74 serotype.

41. The method of any one of claims 1-39, wherein the rAAV is an AAV5 serotype.

42. The method of any one of claims 1-41, wherein the nucleic acid molecule further comprises a Kozak sequence.

43. The method of any one of claims 1-42, wherein the nucleic acid molecule further comprises an miR-122 binding site.

44. A method, comprising:
administering to a subject about 10¹² vector genomes per kilogram (vg/kg) to about 10¹⁴ vg/kg of a recombinant adeno-associated virus 5 (rAAV5) vector, wherein the rAAV5 vector comprises:
(i) a nucleic acid molecule comprising at least one AAV inverted terminal repeat (ITR), and
(ii) a non-AAV nucleotide sequence encoding the amino acid sequence of SEQ ID NO: 1, wherein the non-AAV nucleotide sequence is operably linked to a promoter that directs expression of the 21OH protein in an adrenal gland cell or an adrenal cortex cell.

45. The method of claim 44, wherein about 10¹³ vg/kg to about 10¹⁴ vg/kg of the rAAV5 vector is administered to the subject.

46. The method of claim 45, wherein about 1.5 ×10¹³ vg/kg of the rAAV5 vector is administered to the subject.

47. The method of claim 45, wherein about 3 ×10¹³ vg/kg of the rAAV5 vector is administered to the subject.

48. The method of claim 45, wherein about 6×10¹³ vg/kg of the rAAV5 vector is administered to the subject.

49. The method of any one of claims 45-48, wherein the rAAV5 vector is administered to the subject intravenously.

50. The method of any one of claims 44-49, wherein the subject has 21-hydroxylase deficiency (21OHD).

51. A method of treating a subject having 21-hydroxylase deficiency (21OHD), comprising:
intravenously administering to the subject about 1.5×10¹³ vector genomes per kilogram (vg/kg) of a recombinant adeno-associated virus 5 (rAAV5) vector, wherein the rAAV5 vector comprises:
(i) a nucleic acid molecule comprising at least one AAV inverted terminal repeat (ITR), and
(ii) a non-AAV nucleotide sequence encoding the amino acid sequence of SEQ ID NO: 1, wherein the non-AAV nucleotide sequence is operably linked to a promoter that directs expression of the 21OH protein in an adrenal gland cell or an adrenal cortex cell,
thereby treating 21OHD in the subject.

52. A method of treating a subject having 21-hydroxylase deficiency (21OHD), comprising:
intravenously administering to the subject about 3×10¹³ vector genomes per kilogram (vg/kg) of a recombinant adeno-associated virus 5 (rAAV5) vector, wherein the rAAV5 vector comprises:
(i) a nucleic acid molecule comprising at least one AAV inverted terminal repeat (ITR), and
(ii) a non-AAV nucleotide sequence encoding the amino acid sequence of SEQ ID NO: 1, wherein the non-AAV nucleotide sequence is operably linked to a promoter that directs expression of the 21OH protein in an adrenal gland cell or an adrenal cortex cell,
thereby treating 21OHD in the subject.

53. A method of treating a subject having 21-hydroxylase deficiency (21OHD), comprising:
intravenously administering to the subject about 6×10¹³ vector genomes per kilogram (vg/kg) of a recombinant adeno-associated virus 5 (rAAV5) vector, wherein the rAAV5 vector comprises:
(i) a nucleic acid molecule comprising at least one AAV inverted terminal repeat (ITR), and
(ii) a non-AAV nucleotide sequence encoding the amino acid sequence of SEQ ID NO: 1, wherein the non-AAV nucleotide sequence is operably linked to a promoter that directs expression of the 21OH protein in an adrenal gland cell or an adrenal cortex cell,
thereby treating 21OHD in the subject.

54. The method of any one of claims 44-53, wherein the promoter is a cytomegalovirus/ß-actin hybrid promoter, PGK promoter, or a promoter specific for expression in an adrenal cortex cell.

55. The method of claim 54, wherein the cytomegalovirus/β-actin hybrid promoter is a CAG, CB6, or CBA promoter.

56. The method of any one of claims 44-55, wherein the promoter comprises or consists of the nucleotide sequence of SEQ ID NO: 2, SEQ ID NO: 3, SEQ ID NO:48, or SEQ ID NO:49.

57. The method of any one of claims 44-56, wherein the ITR is an AAV1, AAV2, AAV3, AAV4, AAV5, AAV6, AAV7, AAV8, AAV9, AAV10, AAV11, AAV12, rh10, or rh74 serotype ITR.

58. The method of any one of claims 44-57, wherein the nucleic acid molecule further comprises a Kozak sequence.

59. The method of any one of claims 44-58, wherein the nucleic acid molecule further comprises an miR-122 binding site.

60. The method of any one of claims 44-59, wherein the subject has the Prader stage IV or V form of 21-hydroxylase deficiency (21OHD).

61. The method of any one of claims 44-59 wherein the subject has congenital adrenal hyperplasia (CAH).

62. The method of claim 61, wherein the CAH is classical CAH.

63. The method of any one of claims 1-62, further comprising administering a therapeutically effective amount of an immunosuppressant to the subject.

64. A method, comprising:
administering to the subject a therapeutically effective amount of a recombinant adeno-associated virus 5 (rAAV5) vector and a therapeutically effective amount of an immunosuppressant,
wherein the rAAV5 comprises a nucleic acid molecule comprising:
(i) at least one AAV inverted terminal repeat (ITR) and
(ii) a non-AAV nucleotide sequence encoding the amino acid sequence of SEQ ID NO: 1, wherein the non-AAV nucleotide sequence is operably linked to a promoter that directs expression of the 21OH protein in an adrenal gland cell or an adrenal cortex cell.

65. The method of claim 64, wherein the subject has 21-hydroxylase deficiency (21OHD).

66. A method of treating a subject having 21-hydroxylase deficiency (21OHD), comprising:
intravenously administering to the subject a therapeutically effective amount of a recombinant adeno-associated virus 5 (rAAV5) vector and a therapeutically effective amount of an immunosuppressant,
wherein the rAAV5 comprises a nucleic acid molecule comprising:
(i) at least one AAV inverted terminal repeat (ITR) and
(ii) a non-AAV nucleotide sequence encoding the amino acid sequence of SEQ ID NO: 1, wherein the non-AAV nucleotide sequence is operably linked to a promoter that directs expression of the 21OH protein in an adrenal gland cell or an adrenal cortex cell,
thereby treating 21OHD in the subject.

67. The method of any one of claims 63-66, wherein the immunosuppressant is administered before, concurrently with, and/or after the administration of the rAAV vector.

68. The method of claim 67, wherein the immunosuppressant is administered before the administration of the rAAV vector.

69. The method of claim 68, wherein the immunosuppressant is administered at least 12 hours before the administration of the rAAV vector.

70. The method of claim 69, wherein the immunosuppressant is administered about 2 days before the administration of the rAAV vector.

71. The method of any one of claims 63-70, wherein the immunosuppressant is a non-glucocorticoid immunosuppressant.

72. The method of any one of claims 63-70, wherein the immunosuppressant is an inhibitor of calcineurin.

73. The method of any one of claims 63-72, wherein the immunosuppressant is ciclosporin, tacrolimus, sirolimus, everolimus, zotarolimus, or any combination thereof.

74. The method of claim 73, wherein the immunosuppressant is tacrolimus.

75. The method of any one of claims 63-74, wherein the immunosuppressant is administered orally.

76. The method of any one of claims 63-75, wherein the therapeutically effective amount of the immunosuppressant is in the range of about 0.01 milligram per kilogram (mg/kg) to about 0.05 mg/kg.

77. The method of claim 76, wherein the therapeutically effective amount of the immunosuppressant is about 0.025 mg/kg.

78. The method of any one of claims 63-77, wherein the therapeutically effective amount of the immunosuppressant is administered twice daily.

79. The method of any one of claims 1-78, comprising administering a therapeutically effective amount of a steroid to the subject.

80. The method of claim 79, wherein the steroid is a mineralocorticoid.

81. The method of claim 79, wherein the steroid is a glucocorticoid.

82. The method of claim 79, wherein the steroid is hydrocortisone.

83. The method of any one of claims 79-82, wherein the steroid is administered before, concurrently with, and/or after administration of the rAAV vector.

84. The method of claim 83, wherein the therapeutically effective amount of the steroid administered to the subject before the administration of the rAAV vector is higher than the therapeutically effective amount of the steroid administered to the subject after the administration of the rAAV.

85. The method of any one of claims 1-84, wherein there is a higher level of cortisol and/or aldosterone in the subject after administration of the rAAV, as compared to before administration of the rAAV.

86. The method of any one of claims 1-85, wherein there is a higher level of cortisol and/or aldosterone in the subject after administration of the rAAV, as compared to a control subject having 21-hydroxylase deficiency (21OHD), who has not been administered the rAAV.

87. The method of any one of claims 1-86, wherein there is a lower level of progesterone, 17-OHP, renin, androstenedione (A4) and/or 11-keto steroids in the subject after administration of the rAAV, as compared to before administration of the rAAV.

88. The method of any one of claims 1-87, wherein there is a lower level of progesterone, 17-OHP, renin, androstenedione (A4) and/or 11-keto steroids in the subject after administration of the rAAV, as compared to a control subject having 21-hydroxylase deficiency (21OHD), who has not been administered the rAAV.

89. The method of any one of claims 1-88, wherein the rAAV vector comprises an expression cassette comprising the nucleic acid sequence of SEQ ID NO: 52.


SEQUENCE LISTING (59 sequences)

SEQ ID NO: 1 - protein, 495 aa, Homo sapiens
MLLLGLLLLLPLLAGARLLWNWWKLRSLHLPPLAPGFLHLLQPDLPIYLLGLTQKFGPIYRLHLGLQDVVVLNSKRTIEEAMVKKWADFAGRPEPLTYKLVSRNYPDLSLGDYSLLWKAHKKLTRSALLLGIRDSMEPVVEQLTQEFCERMRAQPGTPVAIEEEFSLLTCSIICYLTFGDKIKDDNLMPAYYKCIQEVLKTWSHWSIQIVDVIPFLRFFPNPGLRRLKQAIEKRDHIVEMQLRQHKESLVAGQWRDMMDYMLQGVAQPSMEEGSGQLLEGHVHMAAVDLLIGGTETTANTLSWAVVFLLHHPEIQQRLQEELDHELGPGASSSRVPYKDRARLPLLNATIAEVLRLRPVVPLALPHRTTRPSSISGYDIPEGTVIIPNLQGAHLDETVWERPHEFWPDRFLEPGKNSRALAFGCGARVCLGEPLARLELFVVLTRLLQAFTLLPSGDALPSLQPLPHCSVILKMQPFQVRLQPRGMGAHSPGQSQ

SEQ ID NO: 2 - DNA, 584 bp, Unknown
  CAG promoter
gcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 3 - DNA, 399 bp, Unknown
  PGK promoter
ccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcc

SEQ ID NO: 4 - DNA, 23 bp, Artificial Sequence
  CYP21A2 transgene primer
acagtcatcattccgaacctcca

SEQ ID NO: 5 - DNA, 23 bp, Artificial Sequence
  CYP21A2 transgene primer
aaggccagagctctggagttctt

SEQ ID NO: 6 - DNA, 23 bp, Artificial Sequence
  mouse brain-derived neurotrophic factor primer
tgctggatgaggaccagaaggtt

SEQ ID NO: 7 - DNA, 22 bp, Artificial Sequence
  mouse brain-derived neurotrophic factor primer
aggaggctccaaaggcacttga

SEQ ID NO: 8 - DNA, 20 bp, Artificial Sequence
  Mc2r primer
tttctcagtcatcttgccga

SEQ ID NO: 9 - DNA, 20 bp, Artificial Sequence
  Mc2r primer
atgctcctctccttggcttt

SEQ ID NO: 10 - DNA, 21 bp, Artificial Sequence
  Prkar1a primer
gcattccttcgggaatacttt

SEQ ID NO: 11 - DNA, 20 bp, Artificial Sequence
  Prkar1a primer
ccctcgagtcagtacggatg

SEQ ID NO: 12 - DNA, 20 bp, Artificial Sequence
  Prkar2a primer
gagtgactcggactcggaag

SEQ ID NO: 13 - DNA, 20 bp, Artificial Sequence
  Prkar2a primer
cctcctcttcttcatcaggg

SEQ ID NO: 14 - DNA, 18 bp, Artificial Sequence
  Prkarca primer
aagaagggcagcgagcag

SEQ ID NO: 15 - DNA, 20 bp, Artificial Sequence
  Prkarca primer
attctgagaaggggtctccc

SEQ ID NO: 16 - DNA, 19 bp, Artificial Sequence
  Prkarcb primer
caagaaaggcagcgaagtg

SEQ ID NO: 17 - DNA, 20 bp, Artificial Sequence
  Prkarcb primer
tcctcaagcccagcattact

SEQ ID NO: 18 - DNA, 20 bp, Artificial Sequence
  Sf1 primer
gccaggagttcgtctgtctc

SEQ ID NO: 19 - DNA, 19 bp, Artificial Sequence
  Sf1 primer
tttcctgggcgtcctttac

SEQ ID NO: 20 - DNA, 20 bp, Artificial Sequence
  Star primer
gggcatactcaacaaccagg

SEQ ID NO: 21 - DNA, 20 bp, Artificial Sequence
  Star primer
gaaacaccttgcccacatct

SEQ ID NO: 22 - DNA, 20 bp, Artificial Sequence
  Cyp11a1 primer
caggccaacattaccgagat

SEQ ID NO: 23 - DNA, 20 bp, Artificial Sequence
  Cyp11a1 primer
ccttcaagttgtgtgccatc

SEQ ID NO: 24 - DNA, 20 bp, Artificial Sequence
  Hsd3b1 primer
gttgtcatccacactgctgc

SEQ ID NO: 25 - DNA, 20 bp, Artificial Sequence
  Hsd3b1 primer
caggcctccaataggttctg

SEQ ID NO: 26 - DNA, 24 bp, Artificial Sequence
  Cyp11b1 primer
atggactttcagtccagtgtgttc

SEQ ID NO: 27 - DNA, 18 bp, Artificial Sequence
  Cyp11b1 primer
gccgctccccaaaaagaa

SEQ ID NO: 28 - DNA, 20 bp, Artificial Sequence
  Cyp17a1 primer
gaagtgctcgtgaagaaggg

SEQ ID NO: 29 - DNA, 20 bp, Artificial Sequence
  Cyp17a1 primer
ctactatccgcaaaggcgac

SEQ ID NO: 30 - DNA, 20 bp, Artificial Sequence
  Cyp11b2 primer
gagacgtggtgtgttcttgc

SEQ ID NO: 31 - DNA, 20 bp, Artificial Sequence
  Cyp11b2 primer
tcccttgctaccatgtccac

SEQ ID NO: 32 - DNA, 23 bp, Artificial Sequence
  CYP21 primer
acagtcatcattccgaacctcca

SEQ ID NO: 33 - DNA, 23 bp, Artificial Sequence
  CYP21 primer
aaggccagagctctggagttctt

SEQ ID NO: 34 - DNA, 20 bp, Artificial Sequence
  Renin primer
ctctgggcactcttgttgct

SEQ ID NO: 35 - DNA, 20 bp, Artificial Sequence
  Renin primer
agaaggcattttcttgagcg

SEQ ID NO: 36 - DNA, 23 bp, Artificial Sequence
  Tbp primer
cccttgtacccttcaccaatgac

SEQ ID NO: 37 - DNA, 28 bp, Artificial Sequence
  Tbp primer
tcacggtagatacaatattttgaagctg

SEQ ID NO: 38 - DNA, 21 bp, Homo sapiens
aaattcgggcccatctacagg

SEQ ID NO: 39 - DNA, 21 bp, Homo sapiens
atggcttcctcaatggtcctc

SEQ ID NO: 40 - DNA, 23 bp, Macaca fascicularis
ctgtcatgctgctgctgagactt

SEQ ID NO: 41 - DNA, 24 bp, Macaca fascicularis
ctttggcatagcattcatgaggat

SEQ ID NO: 42 - DNA, 23 bp, Mus musculus
tgctggatgaggaccagaaggtt

SEQ ID NO: 43 - DNA, 22 bp, Mus musculus
aggaggctccaaaggcacttga

SEQ ID NO: 44 - RNA, 7 nt, Unknown
  Kozak sequence
accaugg

SEQ ID NO: 45 - RNA, 13 nt, Unknown
  Kozak sequence
gccgccaccaugg

SEQ ID NO: 46 - RNA, 8 nt, Unknown
  Kozak sequence
ccaccaug

SEQ ID NO: 47 - RNA, 9 nt, Unknown
  Kozak sequence
ccaccaugg

SEQ ID NO: 48 - DNA, 260 bp, Unknown
  CB6 Promoter
ccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcggg

SEQ ID NO: 49 - DNA, 282 bp, Unknown
  CBA Promoter
tggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgg

SEQ ID NO: 50 - DNA, 1488 bp, Artificial Sequence
  Codon Optimized CYP21a2 gene sequence
atgctgctgctggggctgctgctgctgctgcctctgctggctggggctcgactgctgtggaactggtggaaactgcggtccctgcacctgccacctctggcaccaggcttcctgcacctgctgcagccagacctgcccatctacctgctgggcctgacccagaagtttggccctatctataggctgcacctgggcctgcaggacgtggtggtgctgaactctaagcgcaccatcgaggaggccatggtgaagaagtgggcagatttcgcaggccggccagagccactgacatacaagctggtgagcagaaattatcctgacctgtccctgggcgattactctctgctgtggaaggcccacaagaagctgacaaggagcgccctgctgctgggcatccgcgactccatggagccagtggtggagcagctgacccaggagttttgcgagaggatgagggcacagcctggaacaccagtggccatcgaggaggagttcagcctgctgacctgctccatcatctgttatctgacatttggcgataagatcaaggacgataacctgatgccagcctactataagtgtatccaggaggtgctgaagacctggagccactggagcatccagatcgtggacgtgatccccttcctgaggttctttcctaatccaggcctgcggagactgaagcaggccatcgagaagagggatcacatcgtggagatgcagctgaggcagcacaaggagtccctggtggcaggacagtggagggacatgatggattacatgctgcagggagtggcacagccatctatggaggagggaagcggacagctgctggagggacacgtgcacatggcagcagtggatctgctgatcggaggaaccgagacaacagccaacacactgagctgggccgtggtgtttctgctgcaccaccctgagatccagcagcggctgcaggaggagctggaccacgagctgggacctggagcaagctcctctagagtgccatacaaggatcgggccagactgcccctgctgaatgccaccatcgccgaggtgctgaggctgcgccccgtggtgcctctggccctgcctcacaggaccacaagaccaagctccatctccggctatgacatcccagagggcaccgtgatcatcccaaacctgcagggagcacacctggacgagacagtgtgggagcggccacacgagttctggcccgatagatttctggagcctggcaagaacagccgggccctggccttcggctgcggagcccgggtgtgcctgggcgagccactggccaggctggagctgttcgtggtgctgacccgcctgctgcaggcctttacactgctgccctccggcgatgccctgccttctctgcagccactgcctcactgctccgtgatcctgaagatgcagccctttcaggtccgcctgcagccaagggggatgggggcacatagtccagggcagtctcagtaa

SEQ ID NO: 51 - DNA, 592 bp, Marmota monax
aatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagctgacgtcctttccatggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctg

SEQ ID NO: 52 - DNA, 3901 bp, Artificial Sequence
  expression cassette
  misc_feature (1)..(130): 5′ ITR
  misc_feature (261)..(642): CMVIE Enhancer
  misc_feature (648)..(925): CBA Promoter
  misc_feature (926)..(1942): Intron
  misc_feature (2003)..(3490): hCYP21 Opt
  misc_feature (3624)..(3679): B-globin PolyA
  misc_feature (3772)..(3901): 3′ ITR
ctgcgcgctcgctcgctcactgaggccgggcgaccaaaggtcgcccgacgcccgggctttgcccgggcggcctcagtgagcgagcgagcgcgcagagagggagtggccaactccatcactaggggttcctctgcgcgctcgctcgctcactgaggccgggcgaccaaaggtcgcccgacgcccgggctttgcccgggcggcctcagtgagcgagcgagcgcgcagagagggagtggccaactccatcactaggggttcctctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgctgctgctggggctgctgctgctgctgcctctgctggctggggctcgactgctgtggaactggtggaaactgcggtccctgcacctgccacctctggcaccaggcttcctgcacctgctgcagccagacctgcccatctacctgctgggcctgacccagaagtttggccctatctataggctgcacctgggcctgcaggacgtggtggtgctgaactctaagcgcaccatcgaggaggccatggtgaagaagtgggcagatttcgcaggccggccagagccactgacatacaagctggtgagcagaaattatcctgacctgtccctgggcgattactctctgctgtggaaggcccacaagaagctgacaaggagcgccctgctgctgggcatccgcgactccatggagccagtggtggagcagctgacccaggagttttgcgagaggatgagggcacagcctggaacaccagtggccatcgaggaggagttcagcctgctgacctgctccatcatctgttatctgacatttggcgataagatcaaggacgataacctgatgccagcctactataagtgtatccaggaggtgctgaagacctggagccactggagcatccagatcgtggacgtgatccccttcctgaggttctttcctaatccaggcctgcggagactgaagcaggccatcgagaagagggatcacatcgtggagatgcagctgaggcagcacaaggagtccctggtggcaggacagtggagggacatgatggattacatgctgcagggagtggcacagccatctatggaggagggaagcggacagctgctggagggacacgtgcacatggcagcagtggatctgctgatcggaggaaccgagacaacagccaacacactgagctgggccgtggtgtttctgctgcaccaccctgagatccagcagcggctgcaggaggagctggaccacgagctgggacctggagcaagctcctctagagtgccatacaaggatcgggccagactgcccctgctgaatgccaccatcgccgaggtgctgaggctgcgccccgtggtgcctctggccctgcctcacaggaccacaagaccaagctccatctccggctatgacatcccagagggcaccgtgatcatcccaaacctgcagggagcacacctggacgagacagtgtgggagcggccacacgagttctggcccgatagatttctggagcctggcaagaacagccgggccctggccttcggctgcggagcccgggtgtgcctgggcgagccactggccaggctggagctgttcgtggtgctgacccgcctgctgcaggcctttacactgctgccctccggcgatgccctgccttctctgcagccactgcctcactgctccgtgatcctgaagatgcagccctttcaggtccgcctgcagccaagggggatgggggcacatagtccagggcagtctcagtaaacgcgtggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 53 - DNA, 130 bp, Artificial Sequence
  5′ ITR
ctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 54 - DNA, 382 bp, Artificial Sequence
  CMVIE Enhancer
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattac

SEQ ID NO: 55 - DNA, 278 bp, Artificial Sequence
  CBA Promoter
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 56 - DNA, 1017 bp, Artificial Sequence
  Intron
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 57 - DNA, 1488 bp, Artificial Sequence; identical to SEQ ID NO: 50
  hCYP21 Opt

SEQ ID NO: 58 - DNA, 56 bp, Artificial Sequence
  B-globin PolyA
aataaaggaaatttattttcattgcaatagtgtgttggaattttttgtgtctctca

SEQ ID NO: 59 - DNA, 130 bp, Artificial Sequence
  3′ ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcag